(19) United States
(12) Patent Application Publication
(10) Pub. No.: US 2024/0189265 A1
(43) Pub. Date: Jun. 13, 2024

(54) COMPOSITIONS AND TECHNIQUES FOR VAGINAL INSERTION

(71) Applicant: Daré Bioscience, Inc., San Diego, CA (US)

(72) Inventors: David Friend, San Diego, CA (US); Jennifer Kiang, San Diego, CA (US); Nicolas J. Pacelli, Cedar Park, TX (US); Mark Walters, San Diego, CA (US)

(21) Appl. No.: 18/292,097

(22) PCT Filed: Jul. 25, 2022
(86) PCT No.: PCT/US2022/038234

§ 371 Date: Jan. 25, 2024

Related U.S. Application Data
(60) Provisional application No. 63/331,749, filed on Apr. 15, 2022.
(60) Provisional application No. 63/285,447, filed on Dec. 2, 2021.
(60) Provisional application No. 63/225,872, filed on Jul. 26, 2021.

(51) Int. Cl. A61K 31/196; A61K 9/00; A61K 9/06; A61K 31/5575; A61K 47/10; A61K 47/12; A61K 47/34; A61K 47/36

(52) U.S. Cl. CPC A61K 31/196; A61K 9/0034; A61K 9/06; A61K 31/5575; A61K 47/10; A61K 47/12; A61K 47/34; A61K 47/36

(57) ABSTRACT

The present disclosure generally relates to compositions and techniques for vaginal insertion. In some embodiments, a composition such as a gel may be applied to the vagina of a subject that is relatively viscous, for example, with a viscosity at room temperature of at least 1.5 million cP. The composition may also contain an active ingredient, e.g., dinoprostone, diclofenac, and/or salts thereof. Compositions having such relatively high viscosities may be useful, for example, to prevent the composition from readily exiting the vagina or degrading too quickly. This may, for example, allow the composition to release the active ingredient over a relatively long period of time to the vagina. In some embodiments, such compositions may be prepared by removing air from the composition to increase its viscosity or cause the composition to form a gel, etc. In addition, certain embodiments as described herein are generally directed to techniques for making or using such compositions, kits including such compositions, or the like.

### RELATED APPLICATIONS

This application claims the benefit of priority to U.S. Provisional Application No. 63/225,872, filed Jul. 26, 2021; U.S. Provisional Application No. 63/285,447, filed Dec. 2, 2021; and U.S. Provisional Application No. 63/331,749, filed Apr. 15, 2022, the entire contents of each of which is incorporated herein by reference in its entirety.

### FIELD

The present disclosure generally relates to compositions and techniques for vaginal insertion.

### BACKGROUND

In subjects having a vagina, the vagina represents an important route for drug administration, e.g., due to its large surface area and blood supply. A variety of materials potentially can be applied to the vagina for delivery to a subject, including both local and systemic delivery. However, materials applied to the vagina may be expelled relatively quickly, e.g., due to gravity, as well as vaginal discharge. In addition, the mucosal layer within the vagina presents a significant challenge for drug entry. Accordingly, improvements in vaginal delivery techniques are needed.

### SUMMARY

The present disclosure generally relates to the treatment of compositions and techniques for vaginal insertion. The subject matter of the present disclosure involves, in some cases, interrelated products, alternative solutions to a particular problem, and/or a plurality of different uses of one or more systems and/or articles.

In one aspect, the present disclosure is directed to a composition. The composition, in one set of embodiments, comprises a poloxamer and a stabilization polymer. The composition may have a viscosity at room temperature of at least 1.5 million cP.

According to another set of embodiments, the composition comprises a poloxamer, and a stabilization polymer. The composition may be made by a process comprising forming a composition comprising the poloxamer and the stabilization polymer, and removing air from the composition.

The composition, in another set of embodiments, comprises a poloxamer and a stabilization polymer. In some embodiments, the composition is made by a process comprising forming a composition comprising the poloxamer and the stabilization polymer, and exposing the composition to a pressure of less than 100 mbar (absolute) for at least 30 minutes.

In one set of embodiments, the composition comprises a poloxamer, a stabilization polymer, and an active ingredient for inducing cervical ripening. In some cases, the composition has a viscosity at room temperature of at least 1.5 million cP.

In another set of embodiments, the composition comprises a poloxamer, a stabilization polymer, and an active ingredient for inducing cervical ripening. In some cases, the composition is made by a process comprising forming a composition comprising the poloxamer, the stabilization polymer, and the active ingredient, and removing air from the composition.

In yet another set of embodiments, the composition comprises a poloxamer, a stabilization polymer, and an active ingredient for inducing cervical ripening. In some cases, the composition is made by a process comprising forming a composition comprising the poloxamer, the stabilization polymer, and the active ingredient, and exposing the composition to a pressure of less than 100 mbar (absolute) for at least 30 minutes.

In still another set of embodiments, the composition is a composition for inducement of cervical ripening. In some cases, at least 90 wt % of the composition consists essentially of a poloxamer, a stabilization polymer, an active ingredient for inducing cervical ripening, and water.

In one set of embodiments, the composition comprises a poloxamer, a stabilization polymer, and an active ingredient for treating dysmenorrhea. In some cases, the composition has a viscosity at room temperature of at least 1.5 million cP.

In another set of embodiments, the composition comprises a poloxamer, a stabilization polymer, and an active ingredient for treating dysmenorrhea. In some cases, the composition is made by a process comprising forming a composition comprising the poloxamer, the stabilization polymer, and the active ingredient, and removing air from the composition.

In yet another set of embodiments, the composition comprises a poloxamer, a stabilization polymer, and an active ingredient for treating dysmenorrhea. In some cases, the composition is made by a process comprising forming a composition comprising the poloxamer, the stabilization polymer, and the active ingredient, and exposing the composition to a pressure of less than 100 mbar (absolute) for at least 30 minutes.

In still another set of embodiments, the composition is a composition for treatment of dysmenorrhea. In some cases, at least 90 wt % of the composition consists essentially of a poloxamer, a stabilization polymer, an active ingredient for treating dysmenorrhea, and water.

In another aspect, the present disclosure is directed to a method. The method, according to one set of embodiments, comprises applying, to the vagina of a subject, a gel having a viscosity at room temperature of at least 1.5 million cP.

In another set of embodiments, the method comprises applying, to a vagina of a subject, a composition comprising a poloxamer and a stabilization polymer. In certain embodiments, the composition, as applied, has a viscosity of at least 1.5 million cP.

In another set of embodiments, the method comprises providing a composition comprising a poloxamer and a stabilization polymer, and removing air from the composition.

The method, in still another set of embodiments, comprises providing a composition comprising a poloxamer and a stabilization polymer, and exposing the composition to a pressure of less than 100 mbar (absolute) for at least 30 minutes to form a gel.

In one set of embodiments, the method comprises applying, to a vagina of a subject, a composition comprising a poloxamer, a stabilization polymer, and an active ingredient for inducing cervical ripening. In some cases, the composition, as applied, has a viscosity of at least 1.5 million cP.

In another set of embodiments, the method comprises providing a composition comprising a poloxamer, a stabilization polymer, and an active ingredient for inducing cervical ripening, and removing air from the composition.

In yet another set of embodiments, the method comprises providing a composition comprising a poloxamer, a stabilization polymer, and an active ingredient for inducing cervical ripening, and exposing the composition to a pressure of less than 100 mbar (absolute) for at least 30 minutes to form a gel.

In one set of embodiments, the method comprises applying, to a vagina of a subject, a composition comprising a poloxamer, a stabilization polymer, and an active ingredient for treating dysmenorrhea. In some cases, the composition, as applied, has a viscosity of at least 1.5 million cP.

In another set of embodiments, the method comprises providing a composition comprising a poloxamer, a stabilization polymer, and an active ingredient for treating dysmenorrhea, and removing air from the composition.

In yet another set of embodiments, the method comprises providing a composition comprising a poloxamer, a stabilization polymer, and an active ingredient for treating dysmenorrhea, and exposing the composition to a pressure of less than 100 mbar (absolute) for at least 30 minutes to form a gel.

Several methods are disclosed herein of administering a subject with a compound for prevention or treatment of a particular condition. It is to be understood that in each such aspect of the disclosure, the disclosure specifically includes, also, the compound for use in the treatment or prevention of that particular condition, as well as use of the compound for the manufacture of a medicament for the treatment or prevention of that particular condition.

In another aspect, the present disclosure encompasses methods of making one or more of the embodiments described herein, for example, a composition as described herein. In still another aspect, the present disclosure encompasses methods of using one or more of the embodiments described herein, for example, a composition for the treatment of as described herein, and other indications.

In some embodiments, the present disclosure provides kits comprising the compositions described herein, where the kit includes an applicator suitable for vaginal application. In some embodiments, the applicator is pre-filled with the compositions described herein. In some embodiments, the applicator is not pre-filled with the compositions described herein.

In some embodiments, the kit includes one or more of instructions for inserting the applicator into the vagina and instructions for inducing cervical ripening, for treating dysmenorrhea, for treating menorrhagia or promoting cervical ripening by applying the applicator filled with the composition or gel.

In some embodiments, the composition is at a temperature of about 4° C. for pre-filling the applicator.

In some aspects, provided herein is a method including for treating menorrhagia, the method including applying, to a vagina of a subject, a composition comprising a poloxamer, a stabilization polymer, and an active ingredient for treating menorrhagia, wherein the composition, as applied, has a viscosity of at least 1.5 million cP. In some embodiments, the active ingredient is diclofenac. In embodiments, the subject has menorrhagia. In some embodiments, the subject is at risk of having menorrhagia. In some embodiments, the subject is human.

In some aspects, provided herein is a method including for treating pelvic pain, the method including applying, to a vagina of a subject, a composition comprising a poloxamer, a stabilization polymer, and an active ingredient for treating menorrhagia, wherein the composition, as applied, has a viscosity of at least 1.5 million cP. In some embodiments, the active ingredient is diclofenac. In some embodiments, the pelvic pain is generalized pelvic pain, acute pelvic pain or chronic pelvic pain. For example, pelvic pain can result from endometriosis, adenomyosis, ovulatory pain, ovarian cyst pain and/or pelvic muscle associated pain. In some embodiments, the subject is experiencing pelvic pain. In embodiments, the subject is at risk of pelvic pain. In embodiments, the subject has generalized pelvic pain. In embodiments, the subject has acute pelvic pain. In embodiments, the subject has chronic pelvic pain. In embodiments, the subject has endometriosis. In embodiments, the subject has adenomyosis. In embodiments, the subject has ovulatory pain. In embodiments, the subject has ovarian cyst pain. In embodiments, the subject has an ovarian cyst. In embodiments, the subject has pelvic muscle associated pain.

In some embodiments, the composition is a gel. In embodiments, the poloxamer includes poloxamer 407. In embodiments, the stabilization polymer is xanthan gum.

In embodiments, the composition has air at no more than 15 vol %.

In embodiments, the method includes applying the composition through an applicator to the vagina.

In aspects, provided herein is a method for treating menorrhagia, the method including providing a composition including a poloxamer, a stabilization polymer, and an active ingredient for treating menorrhagia; and removing air from the composition. In embodiments, the active ingredient is diclofenac.

In some embodiments, removing air includes exposing the composition to a pressure of less than 100 mbar (absolute). In some embodiments, removing air includes exposing the composition to a pressure of less than 50 mbar (absolute). In some embodiments, removing air includes exposing the composition to a pressure of less than 40 mbar (absolute).

In some embodiments, the method includes exposing the composition to the pressure for at least 30 min. In embodiments, removing air includes centrifuging the composition at 100 RPM, for example centrifuging the composition for at least 30 min.

In some embodiments, the method includes removing air such that the composition has no more than 15 vol % air.

In some embodiments, the composition has a viscosity at room temperature of at least 1.5 million cP. In some embodiments, the poloxamer is poloxamer 407. In embodiments, the stabilization polymer is xanthan gum.

In embodiments, provided herein is a method for treating menorrhagia, the method including providing a composition including a poloxamer, a stabilization polymer, and an active ingredient for treating menorrhagia; and exposing the composition to a pressure of less than 100 mbar (absolute) for at least 30 minutes to form a gel.

In embodiments, the active ingredient is diclofenac.

In some embodiments, the gel has a viscosity at room temperature of at least 1.5 million cP.

In some embodiments, the method includes exposing the composition to the pressure of less than 100 mbar until the gel comprises no more than 15 vol % air.

In some embodiments, the poloxamer is poloxamer 407. In some embodiments, the stabilization polymer is xanthan gum.

In aspects, provided herein is a method for treating menorrhagia, the method including providing a composition having a poloxamer, a stabilization polymer, and an active ingredient for treating menorrhagia; and exposing the composition to a pressure of less than 100 mbar (absolute) for at least 30 minutes to form a gel.

In some embodiments, the active ingredient is diclofenac.

In some embodiments, the gel has a viscosity at room temperature of at least 1.5 million cP.

In some embodiments, the method includes exposing the composition to the pressure of less than 100 mbar until the gel has no more than 15 vol % air.

In some embodiments, the poloxamer is poloxamer 407. In some embodiments, the stabilization polymer is xanthan gum.

Also provided herein are methods for treating menorrhagia in a subject including comprising administering any of the compositions described herein to the vagina of the subject.

Other advantages and novel features of the present disclosure will become apparent from the following detailed description of various non-limiting embodiments of the disclosure when considered in conjunction with the accompanying figures.

### BRIEF DESCRIPTION OF THE DRAWINGS

Non-limiting embodiments of the present disclosure will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1 illustrates the viscosity of a composition as a function of temperature, in accordance with one embodiment as described herein.

FIG. 2 is a graph showing in vitro release test (IVRT) comparison of drug release versus time (SQRT: square root).

FIG. 3 is a graph showing a regression model for release rate versus drug load.

FIG. 4 is a graph showing a Release plot of a 1% gel composition.

FIG. 5 is a graph showing a linear time plot of a 1% gel composition.

FIG. 6 is a graph showing a Release plot of a 3% gel composition.

FIG. 7 is a graph showing a linear time plot of a 3% gel composition.

FIG. 8 is an image showing appearance results for Batch 178003-2201 (1% diclofenac; Left); Batch 178003-2202 (2% diclofenac; Middle); Batch 178003-2203 (3% diclofenac; Right).

FIG. 9 are microscopy images at 200× magnification for Batch 178003-2201 (1% diclofenac; Left); Batch 178003-2202 (2% diclofenac; Middle); Batch 178003-2203 (3% diclofenac; Right). The images indicated that the API (active pharmaceutical ingredient) is angular crystalline rod-shape material with low sphericity. Batches 178003-2202 & 178003-2203 exhibited higher amounts of API agglomeration in the drug product. There was no indication of agglomeration reported in visual observations.

FIG. 10 is a graph showing a thermal viscosity profile comparison.

FIG. 11A is a graph showing the thermal ramp at a 1% gel composition.

FIG. 11B is a table showing the data from the thermal ramp graph of FIG. 11A.

FIG. 12A is a graph showing the thermal ramp at a 2% gel composition.

FIG. 12B is a table showing the data from the thermal ramp graph of FIG. 12A.

FIG. 13A is a graph showing the thermal ramp at a 3% gel composition.

FIG. 13B is a table showing the data from the thermal ramp graph of FIG. 13A.

### DETAILED DESCRIPTION

The present disclosure generally relates to compositions and techniques for vaginal insertion. In some embodiments, a composition such as a gel may be applied to the vagina of a subject that is relatively viscous, for example, with a viscosity at room temperature of at least 1.5 million cP. The composition may also contain an active ingredient, e.g., dinoprostone, diclofenac, and/or salts thereof. Compositions having such relatively high viscosities may be useful, for example, to prevent the composition from readily exiting the vagina or degrading too quickly. This may, for example, allow the composition to release the active ingredient over a relatively long period of time to the vagina. In some embodiments, such compositions may be prepared by removing air from the composition to increase its viscosity or cause the composition to form a gel, etc. In addition, certain embodiments as described herein are generally directed to techniques for making or using such compositions, kits including such compositions, or the like.

Contrary to compositions for dermal or surface applications, the compositions described herein are formulated for vaginal application (e.g., a mucosal surface). The vaginal epithelium has completely different biophysical properties compared to other skin surfaces. For example, the inner lining of the vagina consists of multiple layers of (squamous) cells. The basal membrane provides the support for the first layer of the epithelium—the basal layer. The intermediate layers lie upon the basal layer, and the superficial layer is the outermost layer of the epithelium. In developing vaginal compositions, additional factors are considered, including viscosity. This is because materials applied to the vagina may be expelled relatively quickly (e.g., due to gravity or vaginal discharge) and thus the viscosity (thickness or stickiness) of the vaginal composition is particularly relevant. Also, the mucosal layer within the vagina presents a unique challenge for drug entry, and the vagina is designed to permit the passage of normal uterine secretions (e.g., flushing of the vaginal discharge). In light of these challenges, developing vaginal compositions presents itself with unique hurdles as compared to developing compositions for other dermal uses. Moreover, advantages of preparing and formulating compositions for vaginal (local) delivery include lower systemic side effects due to lower systemic concentrations of the drug, but equivalent or better symptom relief due to local delivery of the drug to the affected tissue.

One aspect as discussed herein is generally directed to compositions having relatively high viscosities, or resistance to flow or deformation. In some embodiments, such compositions may have viscosities of, for example, at least 1 million cP, 3 million cP, or more, or other viscosities as discussed in more detail below. Fluids with such relatively high viscosities do not flow easily and may even resist flow due to gravity in some cases.

The compositions may contain a polymer, which can increase the viscosity of the composition. For example, the polymer may include a poloxamer, which may form a gel in some cases. In addition, in some cases, the composition may also contain xanthan gum, e.g., that can also act as a stabilizer or a thickening agent, which may help increase viscosity. The composition may also contain an active ingredient, such as dinoprostone, diclofenac, etc., which can be released from the composition over a suitable period of time. The composition may be applied to the vagina or another suitable body cavity of a subject, for example, where release of the active ingredient is desired, e.g., to treat or prevent an indication, such as described herein. More details of these and other compositions, in accordance with various embodiments, are provided below.

Such compositions containing relatively high viscosities may be particularly useful, in certain embodiments, to prevent the composition from readily exiting the vagina (or other cavity), and/or from degrading too quickly after application. This may be used, for example, to assist the delivery of an active ingredient to the subject, e.g., to the vagina of the subject. For instance, fluids with relatively high viscosities may release the active ingredient more slowly and/or uniformly, thereby allowing the active ingredient to be delivered to the subject over a longer period of time. Higher viscosities may also better resist the natural function of the vagina to discharge. Thus, as discussed in more detail herein, the active ingredient may be delivered, e.g., at suitably effective concentrations or amounts, for example, over a period of at least a day, a week, or even longer in some cases. In addition, in some embodiments, only a single dose of the composition may be required to treat a subject, e.g., since the composition does not readily exit the vagina.

As a non-limiting example, in one set of embodiments, a composition may be delivered to a subject, such as a pregnant subject, to include cervical ripening in the subject. In some cases, the composition may be inserted into the vagina, and an active ingredient such as dinoprostone may be delivered, e.g., to the vagina. This may be useful, for example, to facilitate or induce cervical ripening, e.g., before or during labor. As another example, in certain embodiments, a composition may be delivered to a subject having or at risk of dysmenorrhea, menstrual cramps, menorrhagia, and/or pelvic pain. The composition may be applied to the vagina of the subject, and an active ingredient, such as diclofenac, may be delivered to the vagina.

Although other techniques for delivering active ingredients to the vagina (or other body cavity) may also involve the use of relatively high viscosities fluids, these fluids are often selected to have lower viscosities at room temperatures (e.g., about 25° C.) and higher viscosities at body temperatures (e.g., 37° C.). For example, they may be a liquid at room temperature, but become a gel at body temperature. In addition, they may not have viscosities as high as 1 million cP or more; as an example, such a composition may have a viscosity of 300,000 cP at room temperature (and be relatively flowable), increasing to only about 800,000 cP at body temperature.

In contrast, the compositions as discussed herein may have relatively high viscosities, for instance, viscosities as high as 1 million cP, 1.5 million cP or more, even at room temperature. It should be noted that such compositions, due to their high viscosities, are actually difficult to manufacture; accordingly, most other techniques will use compositions with either lower viscosities, or viscosities that are at least low at room temperatures, typically well below 1 million cP. However, without wishing to be bound by any theory, it is believed that having a high initial viscosity, e.g., upon application to the vagina, may be surprisingly useful for rapid symptom relief. A composition with a relatively high viscosity is less able to lose the active ingredient, i.e., prior to application to the vagina, and thus retains the active ingredient for release to the subject more readily than a liquid or less viscous composition. For example, the composition may at least partially seal the vagina better, e.g., to promote treatment with the active ingredient. In addition, a composition with a relatively high viscosity may itself also stay within the vagina longer. Furthermore, in some embodiments, the presence of the gel itself may be beneficial, i.e., even without the presence of an active ingredient such as dinoprostone, diclofenac, etc. Thus, it should be understood that an active ingredient is not always required. Accordingly, such compositions may produce better symptom resolution, e.g., as compared to formulations with relatively low viscosities at room temperature.

Such high viscosities can be achieved, in various embodiments, using techniques such as removing air from the composition, which may increase its viscosity and/or cause it to form a gel. Other techniques may also be used, including any of those described herein. For example, in certain embodiments, a composition may be prepared, e.g., comprising poloxamer, an active ingredient, xanthan gum, water, etc., and the composition treated to remove air from the composition, for example, to reduce the composition to 15 vol % air, or less. Techniques for removing air include, but are not limited to, a variety of techniques, such as centrifugation or exposure to relatively high vacuums, e.g., less than 100 mbar. In addition, it should be understood that the composition need not be limited to those described above, and in other embodiments, other compositions may also be used. Examples of such compositions follow.

For instance, in some aspects, a composition may include one or more poloxamers, xanthan gum, and/or another stabilization polymer, and an active ingredient such as any of those discussed herein. Water may be present in some cases, e.g., such that the composition is a gel, and/or has a relatively high viscosity at room temperature, such as is described herein. Other components may be present as well in certain embodiments, for instance, citrate and/or a citrate salt, benzyl alcohol, or the like. These may act, for example, as excipients, preservatives, antimicrobials, bulking agents, stabilizers, antioxidants, buffers, pH regulating agents, or the like. In addition, in some cases, other components that increase the viscosity of the composition may also be used, for example, hyaluronic acid, alginic acid, modified celluloses such as hydroxypropyl methylcellulose, in addition or instead of poloxomer.

As mentioned, the composition may include one or more poloxamers in one set of embodiments. The poloxamer may be used to increase the viscosity of the composition, e.g., as described herein. In some cases, sufficient poloxamer may be present to cause the composition to form a gel, e.g., at room temperature (about 25° C.) and/or body temperature (about 37° C.). Furthermore, in some cases, the composition may have a gelling temperature, but the gel temperature may be in a range that is physiologically irrelevant. For instance, the gelling temperature may be above 40° C., or below 25° C., and thus, the composition does not change phase or gel at normal physiological or body temperatures.

Poloxamers generally include any of a variety of polyoxyethylene-polyoxypropylene triblock copolymers. In some cases, the poloxamer may be a nonionic block copolymer composed of a central hydrophobic chain of polyoxypropylene (polypropylene oxide) flanked by two hydrophilic chains of polyoxyethylene (polyethylene oxide). In some embodiments, the poloxamers may be soluble in water and other polar and non-polar solvents.

Because the lengths of the polymer blocks can be independently customized, many different poloxamers exist that have slightly different properties. For example, the polxoxamer may have a structure:

HO—[CH₂—CH₂—O]ₐ—[CH₂—CH(CH₃)—O]_{b}—[CH₂—CH₂—O]ₐ—H.

The structure includes a core of propylene oxide (represented by “b” in the above figure), flanked by ethylene oxide subunits (represented by “a” in the above figure), typically on both sides. The sum of the two a's may be, for example, from 50 to 500, from 100 to 300, from 150 to 250, or 200. As another example, a may be between 99 and 103, e.g., 101. In embodiments, b may be, for example, from 30 to 100, from 50 to 80, from 60 to 70, or 65. As another example, b may be between 54 and 58, e.g., 56.

In some embodiments, the ethylene oxide subunits forming the poloxamer may be in molar excess to the propylene oxide subunits. For example, in certain embodiments, the ratio of ethylene oxide subunits to propylene oxide subunits (i.e., a:b) may be, for example, from 3:1 to 5:1, or from 2:1 to 4:1.

Several suitable poloxamers can be readily obtained commercially, including poloxamer 407, Pluronic® F-127, or the like. The composition may include a single poloxamer, or more than one type of poloxamer. In some cases, at least 50 wt %, at least 60 wt %, at least 70 wt %, at least 80 wt %, or at least 90 wt % of the poloxamer within the composition is a single type of poloxamer, for example, poloxamer 407 or Pluronic® F-127.

The molecular weight of the poloxamer may be, in one embodiment, from 5 kDa to 25 kDa. In some instances, the molecular weight of the copolymer may be from 9 kDa to 16 kDa. In some cases, the molecular weight of the poloxamer may be at least 1 kDa, at least 2 kDa, at least 3 kDa, at least 4 kDa, at least 5 kDa, at least 7 kDa, at least 9 kDa, at least 10 kDa, at least 15 kDa, at least 16 kDa, at least 20 kDa, at least 25 kDa, at least 50 kDa, etc. In addition, in certain embodiments, the molecular weight of the poloxamer may be no more than 50 kDa, no more than 25 kDa, no more than 20 kDa, no more than 16 kDa, no more than 15 kDa, no more than 10 kDa, no more than 9 kDa, no more than 5 kDa, no more than 4 kDa, no more than 3 kDa, no more than 2 kDa, no more than 1 kDa, etc. Combinations of any of these are also possible. For instance, the poloxamer may have a molecular weight of between 10 kDa and 15 kDa. As other non-limiting examples, the molecular weight may be between 3 kDa and 5 kDa, between 2 kDa and 4 kDa, between 5 kDa and 20 kDa, between 9 kDa and 16 kDa, etc. The molecular weight, in some cases, may be determined as a weight average molecular weight.

In certain embodiments, the poloxamer may be present within the composition at concentrations of at least 1 wt %, at least 2 wt %, at least 3 wt %, at least 5 wt %, at least 7 wt %, at least 10 wt %, at least 15 wt %, at least 20 wt %, at least 25 wt %, at least 30 wt %, at least 40 wt %, or at least 50 wt %. In addition, in some embodiments, the poloxamer may be present within the composition at concentrations of no more than 50 wt %, no more than 40 wt %, no more than 30 wt %, no more than 25 wt %, no more than 20 wt %, no more than 15 wt %, no more than 10 wt %, no more than 7 wt %, no more than 5 wt %, no more than 3 wt %, no more than 2 wt %, etc. Combinations of any of these are also possible in other embodiments. For example, the poloxamer may be present in a composition at between 10 wt % and 20 wt %, between 5 wt % and 15 wt %, between 15 wt % and 30 wt %, etc.

In one embodiment, the poloxamer used in the composition is Pluronic® F-127. In Pluronic® F-127, the sum of two a's in the above block polymer structure may be 200, and b may have a value of 65. In Pluronic® F-127, the ratio of the sum of two a's to b in the poloxamer (i.e., a:b) may be from 2:1 to 4:1. Tables 1 and 2 illustrate chemical composition and specifications of Pluronic® F-127.

TABLE 1
Chemical Composition of Pluronic ® F-127

| | | | | | | Number average | | |
|---|---|---|---|---|---|---|---|---|
| | | | | Content of | | molecular | Poly- | Number of |
| | | | | Oxyethylene | Molecular | weight | dispersity | repeating |
| Pluranic | Poloxamer | a | b | (Percent) | Weight | (g/mol) | index | units |
| F 127 NF | 407 | 101 | 56 | 71.5-74.9 | 9840-14600 | 10000 | 1.2 | EG₂₀₀PG₆₅PH₆ |

TABLE 2
Specifications of Pluronic ® F-127

| | pH | Cloud | | | | | Ethylene | Propylene | 1,4 |
|---|---|---|---|---|---|---|---|---|---|
| Physical | (2.5% | point, | APHA | | BHT, | Unsaturation | Oxide, | Oxide, | dioxane, |
| Form | in water) | 10% | color | % H₂O | ppm | mEq/g | ppm | ppm | ppm |
| Sold | 5.0-7.5 | >100° C. | 120 | Cast solid 0.4 max. | 50-125 | 0.048 ± 0.017 | 1 max. | 5 max. | 0.002% |
| | | | max. | Prill 0.75 max. | | | | | max. |

In addition, in one set of embodiments, the composition may comprise xanthan gum, and/or another stabilization polymer. Examples of other stabilization polymers include hyaluronic acid, alginic acid, modified celluloses such as hydroxypropyl methylcellulose, or others such as described herein. Xanthan gum generally refers to a high molecular weight polysaccharide used as a food additive and rheology modifier, as would be known by those of ordinary skill in the art. In addition, many such xanthan gums are readily available commercially. Xanthan gum may be produced, as a non-limiting example, by a process involving fermentation of glucose or sucrose by the Xanthomonas campestris bacterium. In some embodiments, the backbone of the polysaccharide chain may have two beta-D-glucose units linked through the 1 and 4 positions. The side chains are formed of two mannose and one glucuronic acid, so the chain has repeating modules of five sugar units. The side chain is linked to every other glucose of the backbone at the 3 position. About half of the terminal mannose units have a pyruvic acid group linked as a ketal to its 4 and 6 positions. The other mannose unit has an acetyl group at the 6 positions. Two of these chains may be aligned to form a double helix, giving a rather rigid rod configuration that accounts for its high efficiency as a viscosifier of water.

However, it should be understood that not all xanthan gums have precisely the above molecular configuration or properties, and that xanthan gums may vary in molecular composition, e.g., depending on the source of the xanthan gum, especially those arising from different biological sources. In addition, other stabilization polymers instead of (or in addition to) xanthan gum can be used, for example, KELTROL® BT and/or KELTROL® RD, KELZAN® XC, KELZAN® XCD, KELZAN® D, KELZAN® CC, XANTURAL® 180, XANTURAL® 75, or the like, all of which can be obtained commercially from various suppliers.

The molecular weight of the xanthan gum or other stabilization polymer can vary. For instance, the xanthan gum or other stabilization polymer may have any suitable molecular weight, for example, at least about 1 million, at least about 2 million, at least about 5 million, at least about 10 million, at least about 25 million, or at least about 50 million. In other embodiments, the molecular weight can vary from about one million to 50 million, e.g., depending upon various factors such as how it is prepared. In some embodiments, the molecular weight can range from approximately 1 million to approximately 25 million, e.g., as measured by a Brookfield Viscometer or other suitable device. In yet other embodiments, the molecular weight may be, for example, 1, 2, 3, 4, or 5 (+/−0.5) million, or 6, 7, 8, 9, 10, 11, 12, 13, 14, 15, 16, 17, 18, 19, 19, 20, 21, 22, 23, 24, or 25 (+/−2) million. Still other molecular weights are also possible.

The xanthan gum (and/or another stabilization polymer) may be present within the composition at concentrations of at least 0.1 wt %, at least 0.2 wt %, at least 0.3 wt %, at least 0.4 wt %, at least 0.5 wt %, at least 0.7 wt %, at least 1 wt %, at least 1.5 wt %, at least 2 wt %, at least 2.5 wt %, at least 3 wt %, at least 3.5 wt %, at least 4 wt %, at least 4.5 wt %, at least 5 wt %, at least 5.5 wt %, at least 6 wt %, at least 6.5 wt %, at least 7 wt %, at least 7.5 wt %, at least 8 wt %, at least 8.5 wt %, at least 9 wt %, at least 9.5 wt %, at least 10 wt %, etc. In addition, in some cases, the xanthan gum and/or other stabilization polymer may be present at no more than 10 wt %, no more than 9.5 wt %, no more than 9 wt %, no more than 8.5 wt %, no more than 8 wt %, no more than 7.5 wt %, no more than 7 wt %, no more than 6.5 wt %, no more than 6 wt %, no more than 5.5 wt %, no more than 5 wt %, no more than 4.5 wt %, no more than 4 wt %, no more than 3.5 wt %, no more than 3 wt %, no more than 2.5 wt %, no more than 2 wt %, no more than 1.5 wt %, no more than 1 wt %, no more than 0.8 wt %, no more than 0.6 wt %, no more than 0.4 wt %, no more than 0.2 wt %, etc. In addition, in certain instances, combinations of any of these ranges are also possible. For example, the xanthan gum and/or other stabilization polymer may be present at between 1 wt % and 5 wt %, between 0.5 wt % and 2 wt %, between 0.5 wt % and 5 wt %, between 0.5 wt % and 2 wt %, or the like.

In one set of embodiments, the composition may also comprise an active ingredient. The active ingredient may be one suitable for treating any of the conditions described herein. For instance, the active ingredient may be one suitable for treatment of a subject for a condition when the active ingredient is delivered to the vagina, or another suitable body cavity. In some embodiments, the active ingredient is present in a therapeutically effective amount. One or more than one active ingredient may be used, depending on the embodiment.

For example, in certain embodiments, the active ingredient may be one that is suitable to facilitate or induce cervical ripening, e.g., before or during labor. In some embodiments, a composition such as is described herein may be inserted into the vagina of a subject, e.g., to induce or facilitate opening of the cervix. In one embodiment, for example, the composition may contain dinoprostone and/or a salt thereof as an active ingredient. However, in other embodiments, other suitable active ingredients include, but are not limited to, prostaglandins such as misoprostol, mifepristone, relaxin, oxytocin, etc., and/or pharmaceutically acceptable salts thereof.

In another set of embodiments, the active ingredient may be one that is suitable to treat a subject having or at risk of dysmenorrhea or menstrual cramps. In some cases, a composition such as is described herein may be inserted into the vagina of a subject. An active ingredient may be released from the composition to treat the subject. In one embodiment, for example, the composition may contain diclofenac and/or a salt thereof as an active ingredient, e.g., diclofenac potassium, diclofenac sodium, etc. However, in other embodiments, other suitable active ingredients include, but are not limited to, ibuprofen, ketoprofen, meclofenamate, mefenamic acid, camylofin, celecoxib, dexibuprofen, estropipate, flurbiprofen, levonorgesterel, naproxen, nimesulide, norgestimate, and ethinyl estradiol, trolamine salicylate, valdecoxib, valethamate, etc., and/or pharmaceutically acceptable salts thereof.

In yet another set of embodiments, the active ingredient may include, but is not limited to, lincomycin, metronidazole, clotrimazole, secnidazole, ornidazole, tinidazole, probiotics, boric acid, etc., and/or pharmaceutically acceptable salts thereof. The pharmaceutical formulation may also include an antibiotic as an active ingredient in certain cases. In another set of embodiments, the active ingredient may be an ingredient that can be delivered to the vaginal, e.g., for local or systemic delivery to the subject. In some embodiments, the active ingredient may be any active ingredient that can be dissolved and/or suspended within a composition as described herein, for example, a gel. For example, the active ingredient may be one that is at least partially water soluble. The gel may be relatively viscous, e.g., as discussed herein. Non-limiting examples of suitable active ingredients include, but are not limited to, lidocaine, hydrocortisone, progesterone, misoprostol, metronidazole, ketoconazole, clobetasol, acyclovir, miconazole, nonoxynol-9, lactobacilli, tinidazole, butoconazole, flucytosine, glycerol monolaurate (GML), or the like.

If a salt is present, the salt may be a pharmaceutically acceptable salt in some embodiments. Pharmaceutically acceptable salts include salts which are, within the scope of sound medical judgment, suitable for use in contact with the tissues of a subject (e.g., a human) without undue toxicity, irritation, allergic response, and the like, and are commensurate with a reasonable benefit/risk ratio. Pharmaceutically acceptable salts may also be salts that are generally safe, non-toxic and neither biologically nor otherwise undesirable and includes that which is acceptable for human pharmaceutical use. Pharmaceutically acceptable salts are well known in the art. Pharmaceutically acceptable salts of the compounds describe herein include those derived from suitable inorganic and organic acids and bases. Examples of pharmaceutically acceptable, nontoxic acid salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, quaternary salts.

In some cases, the active ingredient may be released from the composition over any suitable period of time. For example, a therapeutically effective amount of the active ingredient may be released from 1 to 21 days. In some instances, a therapeutically effective amount of the active ingredient may be released up to about 1 to 7 days, about 5 or 15 days, or about 8 to 29 days following administration. As still other examples, the active ingredient may be released at therapeutically effective amounts from the composition, following application to the subject, for at least 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 or more days. In some embodiments, only a single application of the composition to the subject may be needed. However, in other embodiments, the composition may be applied to the subject more than once, for example, in applications separated by any of the lengths of time discussed herein.

For example, in some cases, tₘₐₓ, or the time at which the maximum concentration of the active ingredient is present in the vagina (due to release by the composition) may be at least 1 day, at least 2 days, at least 3 days, etc., or other times such as those described above. In addition, in some embodiments, tₘₐₓ may be less than 30, 29, 28, 27, 26, 25, 24, 23, 22, 21, 20, 19, 18, 17, 16, 15, 14, 13, 12, 11, 10, 9, 8, 7, 6, 5, 4, 3, or 2 days. Combinations of any of these are also possible, e.g., tₘₐₓ may be between 1 and 3 days, or between 4 and 6 days, etc. Without wishing to be bound by any theory, it is believed that this may be due to the relatively high viscosity of the composition and/or slower release kinetics from the composition.

The active ingredient may be present within the composition at any suitable concentration. For example, the active ingredient may be present at at least 0.1 wt %, at least 0.2 wt %, at least 0.3 wt %, at least 0.4 wt %, at least 0.5 wt %, at least 0.7 wt %, at least 1 wt %, at least 1.5 wt %, at least 2 wt %, at least 2.5 wt %, at least 3 wt %, at least 3.5 wt %, at least 4 wt %, at least 4.5 wt %, at least 5 wt %, at least 5.5 wt %, at least 6 wt %, at least 6.5 wt %, at least 7 wt %, at least 7.5 wt %, at least 8 wt %, at least 8.5 wt %, at least 9 wt %, at least 9.5 wt %, at least 10 wt %, etc. In addition, in some cases, the active ingredient may be present at no more than 10 wt %, no more than 9.5 wt %, no more than 9 wt %, no more than 8.5 wt %, no more than 8 wt %, no more than 7.5 wt %, no more than 7 wt %, no more than 6.5 wt %, no more than 6 wt %, no more than 5.5 wt %, no more than 5 wt %, no more than 4.5 wt %, no more than 4 wt %, no more than 3.5 wt %, no more than 3 wt %, no more than 2.5 wt %, no more than 2 wt %, no more than 1.5 wt %, no more than 1 wt %, no more than 0.8 wt %, no more than 0.6 wt %, no more than 0.4 wt %, no more than 0.2 wt %, etc. In addition, in certain instances, combinations of any of these ranges are also possible. For example, the active ingredient may be present at between 1 wt % and 5 wt %, between 0.5 wt % and 2 wt %, between 0.5 wt % and 5 wt %, or the like.

In addition, in accordance with certain embodiments, the composition may release the active ingredient over an extended period of time. In some cases, this may be determining by determining a concentration of an active ingredient in the mucus of the vagina after a certain period of time, for instance, after 1 day, 2 days, 3 days, etc. For example, in one set of embodiments, the concentration of the active ingredient may be at least 100 micrograms/g, at least 200 micrograms/g, at least 300 micrograms/g, at least 400 micrograms/g, at least 500 micrograms/g, at least 600 micrograms/g, at least 700 micrograms/g, etc. The mucus may be sampled, for example, using a swab, or other techniques known to those of ordinary skill in the art.

In addition, in accordance with certain embodiments, the composition may release the active ingredient over an extended period of time. In some cases, this may be determining by determining a concentration of an active ingredient in the mucus of the vagina after a certain period of time, for instance, after 1 day, 2 days, 3 days, etc. For example, in one set of embodiments, the concentration of the active ingredient may be at least 100 micrograms/g, at least 200 micrograms/g, at least 300 micrograms/g, at least 400 micrograms/g, at least 500 micrograms/g, at least 600 micrograms/g, at least 700 micrograms/g, etc. The mucus may be sampled, for example, using a swab, or other techniques known to those of ordinary skill in the art.

Other components may be present as well within a composition. As a non-limiting example, in one set of embodiments, the composition may include citrate and/or a citrate salt. These may include, for example, citric acid, citric acid monohydrate, sodium citrate, sodium citrate dihydrate, or the like. Other examples include other suitable salts, e.g., to make citrate buffer such as sodium phosphate, potassium phosphate, or the like. Buffers such as these may be used, for example, to maintain the pH of the composition (for example, at around 4.5, or another suitable pH). As another example, the composition may include benzyl alcohol. Benzyl alcohol may be useful, for example, as a solvent or a preservative.

Components such as these may each independently be present in any suitable amount or concentration. For example, a component may be present at at least 0.1 wt %, at least 0.2 wt %, at least 0.3 wt %, at least 0.4 wt %, at least 0.5 wt %, at least 0.7 wt %, at least 1 wt %, at least 1.5 wt %, at least 2 wt %, at least 2.5 wt %, at least 3 wt %, at least 3.5 wt %, at least 4 wt %, at least 4.5 wt %, at least 5 wt %, at least 5.5 wt %, at least 6 wt %, at least 6.5 wt %, at least 7 wt %, at least 7.5 wt %, at least 8 wt %, at least 8.5 wt %, at least 9 wt %, at least 9.5 wt %, at least 10 wt %, etc. In addition, in some cases, a component may be present at no more than 10 wt %, no more than 9.5 wt %, no more than 9 wt %, no more than 8.5 wt %, no more than 8 wt %, no more than 7.5 wt %, no more than 7 wt %, no more than 6.5 wt %, no more than 6 wt %, no more than 5.5 wt %, no more than 5 wt %, no more than 4.5 wt %, no more than 4 wt %, no more than 3.5 wt %, no more than 3 wt %, no more than 2.5 wt %, no more than 2 wt %, no more than 1.5 wt %, no more than 1 wt %, no more than 0.8 wt %, no more than 0.6 wt %, no more than 0.4 wt %, no more than 0.2 wt %, etc. In addition, in certain instances, combinations of any of these ranges are also possible. For example, the component may be present at between 1 wt % and 5 wt %, between 0.5 wt % and 2 wt %, between 0.5 wt % and 5 wt %, between 0.5 wt % and 2 wt %, or the like.

In addition, in one set of embodiments, water may be present within the composition. Any suitable amount of water may be present, for example, such that the composition forms a gel, has a relatively high viscosity as discussed herein, or the like. For example, in some cases, at least 50 wt %, at least 55 wt %, at least 60 wt %, at least 65 wt %, at least 70 wt %, at least 75 wt %, at least 80 wt %, at least 85 wt %, at least 90 wt %, or at least 95 wt % of the composition may be water.

In one set of embodiments, the composition is a gel. The can may be semi-solid material that includes a relatively large amount or concentration of water, e.g., as noted above. In some cases, the polymer (e.g., one or more poloxamers) may from a scaffold structure that contains the water within the gel.

The gel or other composition, e.g., as described herein, may have a relatively high viscosity, at least in one set of embodiments. Those of ordinary skill in the art will be aware of techniques for determining viscosity of a sample, for example, using devices such as a rheometers, viscometers, etc.

In some cases, the composition may have a viscosity at room temperature of at least 1 million cP, at least 1.1 million cP, at least 1.2 million cP, at least 1.3 million cP, at least 1.4 million cP, at least 1.5 million, at least 1.6 million cP, at least 1.8 million cP, at least 2 million cP, at least 2.2 million cP, at least 2.4 million cP, at least 2.6 million cP, at least 2.8 million cP, at least 3 million cP, at least 3.5 million cP, at least 4 million cP, etc. In addition, in certain embodiments, the composition may have a viscosity of no more than 4 million cP, no more than 3.5 million cP, no more than 3 million cP, no more than 2.8 million cP, no more than 2.6 million cP, no more than 2.4 million cP, no more than 2.2 million cP, no more than 2.0 million cP, no more than 1.8 million cP, no more than 1.6 million cP, no more than 1.5 million cP, no more than 1.4 million cP, no more than 1.3 million cP, no more than 1.2 million cP, no more than 1.1 million cP, no more than 1.0 million cP, etc. Combinations of any of these are also possible, for example, the composition may exhibit a viscosity of between 1.5 million cP and 2 million cP, between 1.8 million cP and 2.4 million cP, between 1.2 million cP and 3 million cP, etc.

In some cases, the composition may contain xanthan gum and/or another stabilization polymer, and a polymer such as a poloxamer, which may cause the composition to have a relatively high viscosity. In some embodiments, the composition may contain no other component that changes the viscosity of said composition at room temperature by more than +/−100,000 centipoise.

Due to the natural acidity of the vagina, vaginal compositions typically aim for a pH of about 4.5. In embodiments, the pH of the composition may be between about 4 and about 7. In embodiments, the pH of the composition may be between about 4 and about 5.5. In embodiments, the pH of the composition may be between about 4 and about 5. In embodiments, the pH of the composition may be between about 4.5 and about 5.5. For example, the composition may have pH about 4, 4.1, 4.2, 4.3, 4.4, 4.5, 4.6, 4.7, 4.8, or 4.9. In embodiments, vaginal compositions as described herein (e.g., using diclofenac) have up to about pH 5.5, for example pH 5.0, 5.1, 5.2, 5.3, 5.4, or 5.5. In embodiments, vaginal compositions as described herein (e.g., using diclofenac) have up to about pH 7, for example pH 5.6, 5.7, 5.8, 5.9, 6.0, 6.1, 6.2, 6.3, 6.4, 6.5, 6.6, 6.7, 6.8, 6.9, or 7.0.

In embodiments, a composition as described herein may release about 50% of the drug within the composition in about 1 hour to about 80 hours. In embodiments, a composition as described herein may release about 50% of the drug within the composition in about 10 hours to about 70 hours. In embodiments, a composition as described herein may release about 50% of the drug within the composition in about 20 hours to about 70 hours. In embodiments, a composition as described herein may release about 50% of the drug within the composition in about 10 hours to about 60 hours. In embodiments, a composition as described herein may release about 50% of the drug within the composition in about 10 hours to about 50 hours. In embodiments, a composition as described herein may release about 50% of the drug within the composition in about 10 hours to about 40 hours. In embodiments, a composition as described herein may release about 50% of the drug within the composition in about 10 hours to about 30 hours. In embodiments, a composition as described herein may release about 50% of the drug within the composition in about 15 hours to about 60 hours. In embodiments, a composition as described herein may release about 50% of the drug within the composition in about 15 hours to about 50 hours. In embodiments, a composition as described herein may release about 50% of the drug within the composition in about 15 hours to about 40 hours. In embodiments, a composition as described herein may release about 50% of the drug within the composition in about 15 hours to about 30 hours. In embodiments, a composition as described herein may release about 50% of the drug within the composition in about 20 hours to about 60 hours. In embodiments, a composition as described herein may release about 50% of the drug within the composition in about 20 hours to about 50 hours. In embodiments, a composition as described herein may release about 50% of the drug within the composition in about 20 hours to about 40 hours. In embodiments, a composition as described herein may release about 50% of the drug within the composition in about 20 hours to about 30 hours. The time may be any value or subrange within the recited ranges.

In embodiments, a composition as described herein may release about 80% of the drug within the composition in about 1 hour to about 80 hours. In embodiments, a composition as described herein may release about 80% of the drug within the composition in about 10 hours to about 70 hours. In embodiments, a composition as described herein may release about 80% of the drug within the composition in about 20 hours to about 70 hours. In embodiments, a composition as described herein may release about 80% of the drug within the composition in about 10 hours to about 60 hours. In embodiments, a composition as described herein may release about 80% of the drug within the composition in about 10 hours to about 50 hours. In embodiments, a composition as described herein may release about 80% of the drug within the composition in about 10 hours to about 40 hours. In embodiments, a composition as described herein may release about 80% of the drug within the composition in about 10 hours to about 30 hours. In embodiments, a composition as described herein may release about 80% of the drug within the composition in about 15 hours to about 60 hours. In embodiments, a composition as described herein may release about 80% of the drug within the composition in about 15 hours to about 50 hours. In embodiments, a composition as described herein may release about 80% of the drug within the composition in about 15 hours to about 40 hours. In embodiments, a composition as described herein may release about 80% of the drug within the composition in about 15 hours to about 30 hours. In embodiments, a composition as described herein may release about 80% of the drug within the composition in about 20 hours to about 60 hours. In embodiments, a composition as described herein may release about 80% of the drug within the composition in about 20 hours to about 50 hours. In embodiments, a composition as described herein may release about 80% of the drug within the composition in about 30 hours to about 60 hours. In embodiments, a composition as described herein may release about 80% of the drug within the composition in about 40 hours to about 50 hours. The time may be any value or subrange within the recited ranges.

In embodiments, a composition as described herein may release about 100% of the drug within the composition in about 1 hour to about 80 hours. In embodiments, a composition as described herein may release about 100% of the drug within the composition in about 10 hours to about 70 hours. In embodiments, a composition as described herein may release about 100% of the drug within the composition in about 20 hours to about 70 hours. In embodiments, a composition as described herein may release about 100% of the drug within the composition in about 10 hours to about 60 hours. In embodiments, a composition as described herein may release about 100% of the drug within the composition in about 10 hours to about 50 hours. In embodiments, a composition as described herein may release about 100% of the drug within the composition in about 10 hours to about 40 hours. In embodiments, a composition as described herein may release about 100% of the drug within the composition in about 10 hours to about 30 hours. In embodiments, a composition as described herein may release about 100% of the drug within the composition in about 15 hours to about 60 hours. In embodiments, a composition as described herein may release about 100% of the drug within the composition in about 20 hours to about 60 hours. In embodiments, a composition as described herein may release about 100% of the drug within the composition in about 30 hours to about 60 hours. In embodiments, a composition as described herein may release about 100% of the drug within the composition in about 40 hours to about 60 hours. In embodiments, a composition as described herein may release about 100% of the drug within the composition in about 45 hours to about 60 hours. The time may be any value or subrange within the recited ranges.

In addition, in one set of embodiments, the composition may have a relatively low concentration or amount of air. For instance, in one embodiment, the composition is substantially free of air. In some cases, during manufacture, a large amount of air may be introduced into the composition, e.g., as foam or bubbles, etc. However, this air may be undesirable in accordance with certain embodiments, and accordingly, the composition may be prepared by also including a step of removing air from the composition that has been introduced during manufacture.

In some embodiments, the composition, after removing at least some of the air may contain no more than 20 vol %, no more than 15 vol %, no more than 12 vol %, no more than 10 vol %, no more than 8 vol %, no more than 6 vol %, no more than 5 vol %, no more than 4 vol %, no more than 3 vol %, no more than 2 vol %, or no more than 1 vol %, etc. of air. In some cases, the air may be removed such that no air bubbles are visually present within the composition.

Without wishing to be bound by any theory, it is believed that the presence of air may reduce the viscosity of the composition, e.g., making it easier for the composition to flow. Accordingly, in some embodiments, any air that is introduced may be removed, thereby increasing the viscosity of the composition, e.g., to at least 1 million cP, or other ranges of viscosities, such as any of those described herein. Furthermore, in some cases, removing the air may also increase the density of the final composition. For instance, the density of the composition may be at least 0.98 g/cm³, at least 0.99 g/cm³, at least 1 g/cm³, at least 1.01 g/cm³, at least 1.02 g/cm³, at least 1.03 g/cm³, at least 1.05 g/cm³, at least 1.1 g/cm³, etc. In some cases, the density of the composition may be no more than 1.1 g/cm³, no more than 1.05 g/cm³, no more than 1.03 g/cm³, no more than 1.02 g/cm³, no more than 1.01 g/cm³, etc. Furthermore, in some embodiments, combinations of any of these ranges are possible. For example, the final density of the composition may be between 1.00 g/cm³ and 1.01 g/cm³, between 0.99 g/cm³ and 1.02 g/cm³, etc.

In one embodiment, air may be removed from a composition, e.g., during or after formation, by applying a pressure less than atmospheric or ambient pressure to the composition. For instance, the pressure that is applied may be less than 1 bar, less than 800 mbar, less than 600 mbar, less than 500 mbar, less than 400 mbar, less than 300 mbar, less than 200 mbar, less than 100 mbar, less than 75 mbar, less than 60 mbar, less than 50 mbar, less than 40 mbar, less than 30 mbar, less than 20 mbar, less than 10 mbar, less than 5 mbar, less than 3 mbar, less than 2 mbar, less than 1 mbar, etc. It should be noted that 1 atmosphere is approximately 1 bar, and that these pressures are absolute pressures (i.e., a pressure of less than about 1 bar means a pressure lower than atmospheric pressure, i.e., a vacuum pressure). Such pressures may be applied for any suitable length of time, e.g., at least 10 min, at least 20 min, at least 30 min, at least 45 min, at least 1 h, at least 2 h, at least 3 h, at least 4 h, at least 6 h, at least 24 h, at least 1 day, etc. In addition, in some cases, the pressure may be applied until the composition comprises less than a certain amount of air, e.g., less than 15 vol %, or other percentages such as those described herein. As yet another example, in some cases, a solution may be caused to form a gel by removing a certain amount of air from the solution.

In certain embodiments, as another example, the air may be removed from the composition using a Versator or other deaerator, degasser, and/or defoamer. In a Versator, a material such as a liquid is spread onto the inside of a rotating Versator disc under vacuum to remove entrapped air, foam, gas, etc. While the liquid travels across the disc, the high vacuum draws off the bubbles, etc., from the liquid. Versators may be obtained from several commercial sources. Accordingly, in some embodiments, a composition such as described herein may be treated using a Versator for any suitable length of time, e.g., at least 10 min, at least 20 min, at least 30 min, at least 45 min, at least 1 h, at least 2 h, at least 3 h, at least 4 h, at least 6 h, at least 24 h, at least 1 day, etc., and/or until the composition comprises less than a certain amount of air, e.g., less than 15 vol %, or other percentages such as those described herein

As yet another example, in some cases, the air may be removed from the composition using centrifugation. Without wishing to be bound by any theory, it is believed that by centrifuging the composition, air (being less dense) may be forced out of the composition. Thus, for example, the material may be centrifuged at any suitable speed, e.g., at least at least 500 RPM, at least 1,000 RPM, at least 2,000 RPM, at least 3,000 RPM, at least 5,000, at least 10,000 RPM, or the like, for any suitable length of time, e.g., at least 10 min, at least 20 min, at least 30 min, at least 45 min, at least 1 h, at least 2 h, at least 3 h, at least 4 h, at least 6 h, at least 24 h, at least 1 day, etc., and/or until the composition comprises less than a certain amount of air, e.g., less than 15 vol %, or other percentages such as those described herein.

Furthermore, it should be understood that these techniques are non-limiting, and that other methods of removing air, besides centrifuges or Versators, are also possible in still other embodiments.

In embodiments, the product can be manually or filled using an automated filling machine. In some embodiments, the automated filling machine (e.g., a Capmatic automated filing machine) is especially designed to fill applicator-based products, such as vaginal applicator-based products. As the viscosity of the product decreased at refrigerated temperatures, e.g., at temperatures of about 4° C. (e.g., a reduction in viscosity of 2-3×), the product may become suitable for automated filling into a vaginal applicator. In some embodiments, a cooled, jacketed vessel, feeding cooled product into the filler hopper to fill the applicators is used.

### Methods of Use

Accordingly, as discussed above, a variety of compositions are covered in various embodiments, including any suitable combinations of any of the above-described components, such as poloxamer, xanthan gum and/or another stabilization polymer, an active ingredient, and water. For example, in one set of embodiments, at least 50 wt %, at least 60 wt %, at least 70 wt %, at least 80 wt %, at least 90 wt %, at least 95 wt %, or at least 99 wt % of the composition comprises or consists essentially of polymer such as a poloxamer, xanthan gum and/or another stabilization polymer, an active ingredient, and water. The active ingredient may include any of those described herein, for example, dinoprostone, diclofenac, etc., as well as salts thereof.

Furthermore, as mentioned, certain aspects as described herein are generally directed to compositions and methods for applying such compositions for the treatment or prevention of indications such as any of those described herein, e.g., to the vagina of a subject, such as a human. The subject can also be a non-human animal. In embodiments, the subject is female.

In other embodiments, indications that can be treated with a composition such as those described herein include, but are not limited to, vulvovaginal candidiasis (yeast infection), vulvovaginal pain, cervical or vaginal cancer, hormone therapy, etc.

In one set of embodiments, a composition such as is described herein may be used to facilitate or induce cervical ripening in a subject, e.g., before or during labor. In a subject about to give birth, the cervix may open or dilate to allow the baby to be born. However, in some cases, cervical ripening does not occur, and/or occurs but at too slow of a rate, and accordingly, some intervention may be required to facilitate or induce cervical ripening. In some cases, a subject may be treated by applying a composition such as is described herein to the vagina, e.g., to facilitate or induce cervical ripening. The composition may have a suitable active ingredient, such as dinoprostone and/or a salt thereof, and/or other active ingredients such as those described herein. After insertion, the active ingredient may be released, e.g., into the vagina or cervix, which may thus facilitate or induce cervical ripening.

In another set of embodiments, a composition such is described herein may be used to treat a subject having or at risk of dysmenorrhea or menstrual cramps. These are often caused by uterine contractions during menstruation. Although some menstrual pain is normal, subjects with dysmenorrhea often experience pain so severe that it prevents them from doing normal activities for several days, often lasting 3, 4, or more days. In some cases, a subject may be treated by applying a composition such as is described herein to the vagina to treat the dysmenorrhea. The composition may have a suitable active ingredient, such as diclofenac and/or a salt thereof, and/or other active ingredients such as those described herein. After insertion, the active ingredient may be released, e.g., into the vagina or cervix to treat or prevent dysmenorrhea.

In another set of embodiments, a composition such as described herein may be used to treat a subject having or at risk of having menorrhagia. Menorrhagia is heavy menstrual bleeding. It is a common disorder in adolescents and reproductive age women and if untreated, can result in anemia, work and school absenteeism and other disruptions to activities of daily living. Dysmenorrhea and menorrhagia are also common side effects of the copper (Cu) intrauterine device (IUD). First line, non-hormonal treatments of menorrhagia include administration of oral non-steroidal anti-inflammatory drugs (NSAIDs), such as diclofenac. Data support that oral NSAIDs reduce menorrhagia by similar mechanisms which also result in reduced dysmenorrhea, including reduction in endometrial leukotrienes and inflammation. Tranexamic acid is also a non-hormonal medication which significantly reduces menorrhagia, however, this non-hormonal agent cannot be used long term due to the risk of thrombosis.

The advantage of oral NSAIDs is that they are generally available over-the-counter and are effective in reducing menorrhagia and dysmenorrhea, even in Cu IUD users. However, common dose-dependent side effects of oral NSAIDs include gastrointestinal bleeding, diarrhea, and nausea. In addition, taking daily oral NSAIDs for several months to manage menorrhagia and or dysmenorrhea and or side effects of Cu IUDs requires high adherence, especially difficult in younger populations and most importantly long term systemic use of NSAIDs can result in renal damage. Oral NSAIDs are also a first line non-hormonal treatment for the management of generalized pelvic pain, acute pelvic pain and chronic pelvic pain, which can result from endometriosis, adenomyosis, ovulatory pain (Mittleschmirtz), ovarian cyst pain and/or pelvic muscle associated pain.

The oral NSAID dose and the frequency of oral dosing are the same for the treatment of menorrhagia, dysmenorrhea, generalized pelvic pain, acute pelvic pain, ovulatory pain, ovarian cyst pain and pelvic muscle associated pain. Oral NSAIDs are most commonly given as needed or “prn” for the treatment of pain. The duration of treatment for oral NSAIDs is generally shorter for acute pelvic pain, ovulatory pain, dysmenorrhea and menorrhagia. Longer, chronic administration of NSAIDs is generally required for chronic conditions such as endometriosis, adenomyosis, and generalized pelvic pain. It is expected that the treatment duration for the diclofenac vaginal hydrogel would follow the same flexible dosing schedule, based on the individual's specific pain levels and causative factors.

### Local Genital Delivery of NSAIDS for Dysmenorrhea and Menorrhagia

Local delivery of active pharmaceutical ingredients through the vaginal mucosa is ideal because lower doses can be used to target local genital tissues and the myometrium. Data consistently support that drugs delivered vaginally concentrate 10-100 times higher in genital tissues and fluids compared to systemic blood concentrations, which reduces systemic side effects. This is particularly important when the expectation is that a patient will need longer term or chronic NSAID therapy for conditions such as endometriosis or generalized pelvic pain.

In another set of embodiments, a composition such as described herein may be used to treat a subject having or at risk of having generalized pelvic pain, acute pelvic pain and chronic pelvic pain. Without wishing to be bound by theory, these can result from endometriosis, adenomyosis, ovulatory pain, ovarian cyst pain and or pelvic muscle associated pain.

Dosing may or may not be different depending on the underlying cause. In some embodiments, the only difference in dosing is the duration of treatment, e.g., where chronic pelvic pain from chronic conditions (such as endometriosis) likely needs longer durations of treatment (e.g. weeks to months) whereas acute pain or pain flares (e.g. dysmenorrhea, ovulatory pain) likely requires a shorter duration of pain. Most of the time oral NSAIDs are dosed as needed, for example, for dysmenorrhea this is normally during the premenstrual time, when uterine contractions are beginning and for maybe the first few days of menses.

In embodiments, the composition is administered to a subject between one and ten times. In embodiments, the composition is administered to a subject between one and ten times per menstrual cycle. In embodiments, the composition is administered to a subject between one and five times per menstrual cycle. In embodiments, the composition is administered to a subject between one and three times per menstrual cycle. In embodiments, the composition is administered to a subject one time per menstrual cycle. In embodiments, the composition is administered to a subject twice per menstrual cycle.

In embodiments, the composition is administered daily. In embodiments, the composition is administered every two days. In embodiments, the composition is administered every 3, 4, 5, 6, 7, 8, 9, 10, 20, 28, or 30 days. In embodiments, the composition is administered as needed, for example when the subject is in pain.

Terms such as “treat,” “treatment,” “treating,” etc. comprise therapeutic treatment of subjects having already developed a disease, in particular in manifest form. Therapeutic treatment may be symptomatic treatment in order to relieve the signs and/or symptoms of the disease or causal treatment in order to reverse, partially reverse, stop, or slow down the progression of the disease. Thus, the compositions and methods of the present disclosure may be used, for instance, as therapeutic treatment (e.g., for acute or chronic therapy).

Additionally, terms such as “prevent,” “preventing,” or “prevention” generally refer to the reduction of the occurrence of the disease, and/or a sign and/or symptom thereof, in the treated sample relative to an untreated control sample, or delays the onset of one or more signs and/or symptoms of the disease relative to the untreated control sample, in a statistically significant manner. Preventing the disease, and/or a sign and/or a symptom thereof, includes preventing or delaying the initiation of the disease, sign, and/or symptom. Prevention also includes preventing a recurrence of the disease, sign, and/or symptom.

In certain aspects, the composition can be applied to a subject, e.g., to the vagina of a subject, and/or to another body cavity, for example, the mouth or the rectum. Any suitable technique may be used to apply the composition to the subject. For instance, the composition may be free or mass flowing, e.g., so that it may be administered through an applicator or other suitable device. Thus, in some embodiments, the composition may be contained within applicator, such as a vaginal applicator or a syringe, which can be applied, e.g., by the subject, or by another person.

The subject may be, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., monkeys such as cynomolgus monkeys or rhesus monkeys, chimpanzees, etc.); commercially relevant mammals such as cattle, pigs, horses, sheep, rabbits, mice, rats, goats, cats, dogs, etc.) and birds (e.g., commercially relevant birds such as chickens, ducks, geese, turkeys, etc.). In certain embodiments, the subject is a mammal. The subject may be a male or female and at any stage of development. A non-human animal may be a transgenic animal.

In one set of embodiments, as discussed, the composition is applied to treat the subject with a therapeutically effective amount of an active ingredient, such as any of those described herein. The therapeutically effective amount may be an amount which, when administered to a subject for treating or preventing a disease, is sufficient to effect such treatment or prevention for the disease, for example, any of those described herein. Examples include, but are not limited to, vulvovaginal candidiasis (yeast infection), vulvovaginal pain, cervical or vaginal cancer, hormone therapy, or the like, e.g., as discussed herein. The therapeutically effective amount may also be an amount sufficient to elicit a desired biological response, i.e., alleviating a symptom. The therapeutically effective amount may vary depending on such factors as the desired biological endpoint, the mode of administration, and/or the age and health of the subject.

### Kits

Still another aspect of the present disclosure is directed to kits. The kit may include a package or an assembly including one or more of the compositions as described herein, and/or other compositions. Each of the compositions of the kit may be provided in liquid form (e.g., in solution), or in solid form (e.g., a dried powder), or in gaseous form in some cases. In certain cases, some of the compositions may be constitutable or otherwise processable (e.g., to an active form), for example, by the addition of a suitable solvent or other species, which may or may not be provided with the kit. Examples of other components include, but are not limited to, solvents, surfactants, diluents, salts, buffers, emulsifiers, chelating agents, fillers, antioxidants, binding agents, bulking agents, preservatives, drying agents, antimicrobials, needles, syringes, packaging materials, tubes, bottles, flasks, beakers, dishes, frits, filters, rings, clamps, wraps, patches, containers, and the like, for example, for using, administering, modifying, assembling, storing, packaging, preparing, mixing, diluting, and/or preserving the compositions components for a particular use, for example, to a sample and/or a subject.

A kit may, in some cases, include instructions in any form that are provided in connection with the compositions described herein in such a manner that one of ordinary skill in the art would recognize that the instructions are to be associated with those compositions. For instance, the instructions may include instructions for the use, modification, mixing, diluting, preserving, administering, assembly, storage, packaging, and/or preparation of the compositions and/or other compositions associated with the kit. In some cases, the instructions may also include instructions for the delivery and/or administration of the compositions, for example, for a particular use, e.g., to a sample and/or a subject. The instructions may be provided in any form recognizable by one of ordinary skill in the art as a suitable vehicle for containing such instructions, for example, written or published, verbal, audible (e.g., telephonic), digital, optical, visual (e.g., videotape, DVD, etc.) or electronic communications (including Internet or web-based communications), provided in any manner.

In an embodiments, provided herein are kits comprising the compositions and gels described herein. For example, the kit includes an applicator suitable for vaginal application. In other examples, the applicator is pre-filled with the compositions or the gels described herein. In other embodiments, the applicator is not-prefilled with the compositions or the gels described herein. In some embodiments, the compositions or gels described herein are at refrigerated temperatures (e.g., about 4° C.) prior to pre-filling the applicator. In some embodiments, the kit includes an applicator that is pre-filled with the composition, where the composition includes a poloxamer, a stabilization polymer, and an active ingredient (e.g., diclofenac or any other therapeutic agent for inducing cervical ripening or treating dysmenorrhea or treating primary dysmenorrhea).

In some embodiments, the kit further includes one or more instructions for inserting the applicator into the vagina. In other embodiments, the kit includes instructions for inducing cervical ripening, treating dysmenorrhea, treating primary dismenorrhea by applying the applicator filled with the compositions or gels described herein. In some embodiments, the kit further includes instructions for filling the applicator with the composition or gel.

In some embodiments, the compositions described herein are stored at room temperature (either pre-filled in an applicator or a separate storage container). In some embodiments, the compositions can be stored for 1 day, 2 days, 10 days, 2 weeks, 1 month, 2 months, 3 months, 4 months, 6 months, 7 months, 8 months, 9 months, 10 months, 11 months, 1 year, or more at room temperature.

### EXAMPLES

The following examples are intended to illustrate certain embodiments of the present disclosure, but do not exemplify the full scope of the disclosure.

### Example 1: Composition

In this prophetic example, a composition in accordance with one embodiment is as follows (all components USP grade). The active ingredient may be dinoprostone, diclofenac, or the like.

TABLE 3
composition

| Material | g/100 g |
|---|---|
| Poloxamer 407 | 16.50 |
| Xanthan gum | 2.00 |
| Active ingredient | 2.00 |
| Citric acid monohydrate | 0.60 |
| Sodium citrate dihydrate | 0.55 |
| Benzyl alcohol | 1.00 |
| Purified water (qs) | 77.35 |
| Total | 100 |

### Example 2: Method of Making the Composition

This prophetic example illustrates a method of making the composition described in Example 1, in accordance with another embodiment.

Purified water is added to a kettle (or other suitable container), which is placed under a dissolver (30-60 HP) with a 12-inch stand dissolver blade. The dissolver is started, and an active ingredient is added to the kettle. The dissolver is allowed to mix for at least 10 minutes, or until the active ingredient has visually dissolved in the water.

Afterwards, mixing is continued while citric acid monohydrate and sodium citrate dihydrate are added to the kettle. The kettle is then cooled with chilled cooling water, e.g., a temperature of 8° C. Mixing is continued for at least 5 minutes, or until these have visually dissolved.

Mixing and cooling then continues while benzyl alcohol and poloxamer 407 is added. These are mixed until the water is clear and everything has visually dissolved.

Mixing and cooling continues while xanthan gum is slowly added. Mixing of the xanthan gum continues for at least 10 minutes, or until the composition is visually uniform. The speed of mixing may be adjusted, for example, as the composition thickens and its viscosity increases, and/or to avoid trapping too many air bubbles within the composition.

Next, the composition is transferred into a round-bottom, jacketed, stainless steel pressure/vacuum kettle, or another suitable container. The kettle is cooled as before, e.g., using chilled cooling water at 8° C. Mixing in the kettle is started and the batch is slowly recirculated under a vacuum. Mixing, cooling with chilled cooling water, and recirculating under vacuum occurs for at least 30 minutes, or until the composition has been sufficiently deaerated.

It should be understood that the above is by way of example only, and that other techniques for making the compositions described herein are also contemplated. For example, the addition sequence of poloxamer 407 and xanthan gum may be reversed, with the poloxamer going into the mixing first. Air may be removed using a centrifuge, a Versator, or the like.

### Example 3: Method of Making the Composition

This prophetic example illustrates a method of making a composition in accordance with another embodiment.

The primary compounding phase in this example is prepared by adding 154 kg of Purified Water USP to a 270 L kettle. The kettle is placed under a dissolver equipped with a 10-inch standard blade. Mixing commences at about 600 rpm. 4.86 kg of an active ingredient is added and mixed for 10 minutes until visually dissolved. Mixing continues and 1200 g citric acid and 1100 g of sodium citrate are added and mixed for 6 min until visually dissolved. Cooling of the batch is started during this step by flowing chilled water through the kettle jacket.

Mixing continues and 2000 g of benzyl alcohol and 33.0 kg of poloxamer 407 are added. Cooling and mixing continue until the solution is clear and the poloxamer is visually dissolved. The batch temperature reaches approximately 11° C. during this step. This step creates foam on the surface of the batch and the mixing speed is decreased to allow the foam to dissipate. Mixing speed is then increased, eventually reaching 1200 rpm for addition of the 4.00 kg of xanthan gum. After the addition of the xanthan gum, the product rapidly increases in viscosity and the original mixing blade size is switched to a 12-inch blade to better facilitate mixing at this stage of the process. A 14-inch blade may also be used at this scale.

After this step in the process, the product has generated a large amount of foaming. The foaming can be reduced by transferring the product into a cooling, jacketed vacuum vessel and recirculate the product for several passes to help remove the air. Another approach is to use a Versator, which is a device for removing air from liquids and semi-solids. The product was passed through the Versator several times until all air was removed. Measurements of the viscosity after Versator treatment rose to over 3 million cP, from 1.2 million cP at room temperature prior to using the Versator.

### Example 4: Viscosity of the Composition

The present example shows the viscosity of a composition in accordance with one embodiment as a function of temperature. It will be noted that, even at room temperature (about 25° C.), the composition has a viscosity of over 1 million cP.

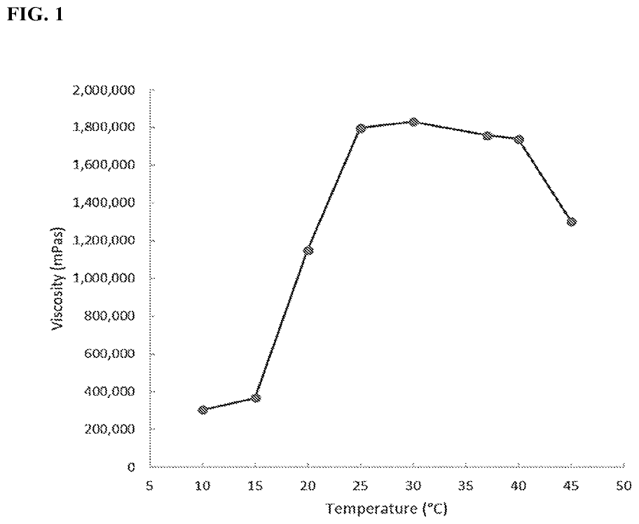

FIG. 1 illustrates the viscosity (in mPA s, equivalent to cP) plotted as a function of temperature for the composition shown in Example 1. Viscosity was measured using a Brookfield Viscometer.

In this experiment, the viscosity at 25° C. was found to be 1,798,000 cP, while the viscosity at 37° C. (body temperature) was found to be 1,759,000 cP.

### Example 5: In Vitro Release Testing (IVRT) Results

In vitro release testing (IVRT) was evaluated for diclofenac sodium gel 1%, 2%, and 3% at n=6 using a PION Rainbow® in situ fiber optic probes with PDA (200-720 nm) with 2 mm stainless steel probes. The results for the average release and release rate (slope) are presented in Table 4 (below).

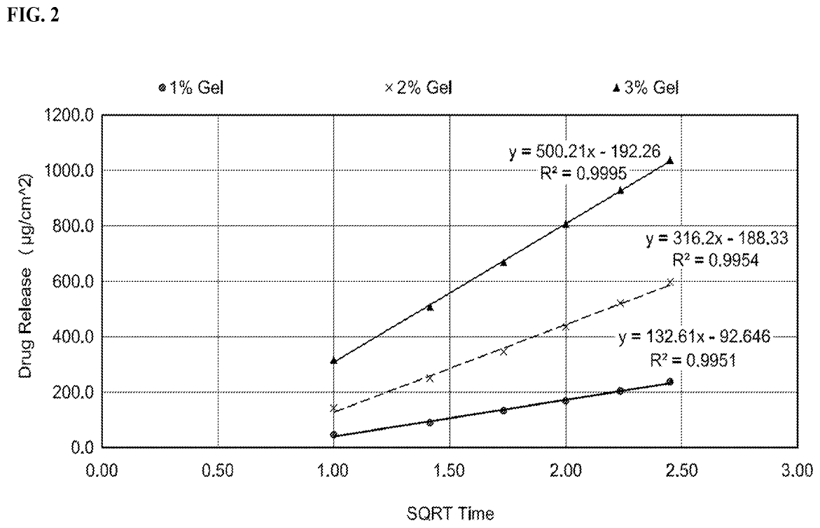

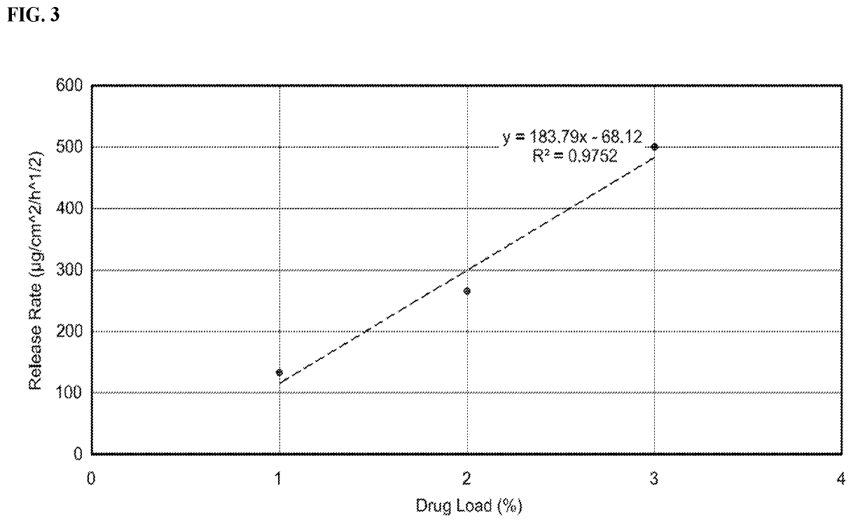

The release rate increased proportionally with the dose (FIG. 2 and FIG. 3). The release rate did not strictly follow the classical Higuchi model for drug release of suspensions from the matrix, which may be explained by the additional changes in the rheology which correlate to the increase solids content in the formulation. The result describes a more complex relationship between the formulation composition and the drug release.

TABLE 4
IVRT Results

| | | |
|---|---|---|
| Time | SQRT | Average Drug Release (μg/cm²) |

| (hours) | Time | 1% Gel | 2% Gel | 3% Gel |
|---|---|---|---|---|
| 1 | 1.00 | 46.4 | 142.5 | 316.6 |
| 2 | 1.41 | 89.9 | 250.0 | 507.5 |
| 3 | 1.73 | 133.6 | 346.6 | 668.4 |
| 4 | 2.00 | 168.9 | 436.7 | 806.6 |
| 5 | 2.24 | 203.9 | 521.1 | 928.3 |
| 6 | 2.45 | 237.9 | 598.3 | 1037.5 |

| | | | |
|---|---|---|---|
| Slope (Rate) | 132.6 | 316.2 | 500.2 |
| R² | 0.995 | 0.995 | 0.999 |
| % RSD | 2.2 | 4.3 | 2.5 |

The release rates obtained during IVRT for the three test batches were used to calculate the estimated time for 50, 80, and 100% drug release (Table 5, below) based on a dose coverage of ˜0.23 g/cm² assuming a constant release rate. Typically the release rate slowed as the dose is depleted so it would be expected that the actual times for 80% and 100% drug release would be longer than provided by this estimate.

There are at least two notable aspects of this release data. First, diclofenac is in suspension, not solution, within the gel. As compared to poloxamer gel containing a solubilized active agent (e.g., clindamycin), diclofenac would be expected to take longer to release out of an equivalent amount of gel matrix. Second, a relationship between pH and diclofenac release rate has been observed. It is implicit in the data in Table 4 showing a non-linear variance in release rate as the API concentration goes up. When combined with the data in Table 17 that the pH goes up as the API concentration goes up, it supports the conclusion that higher pH supports faster release. Table 5 further supports this by showing that the 3% gel releases 50%, 80% and 100% faster than 1% or 2% diclofenac compositions.

TABLE 5
Estimated time for diclofenac release

| | Time (hours) | | |
|---|---|---|---|
| | 50% Drug | 80% Drug | 100% Drug |
| Sample | Release | Release | Release |
| 1% Gel | 30 | 47 | 59 |
| 2% Gel | 25 | 40 | 50 |
| 3% Gel | 24 | 40 | 47 |

### IVRT for Diclofenac Gel at 1% and 3% w/w

Additional IVRT was performed for diclofenac vaginal gel 1% and 3% w/w. For this analysis the receptor media was changed from phosphate buffer pH 7 to simulated vaginal fluid. Sampling was executed every 60 minutes for 6 hours, and a single point at T=24 hours. Total volume replacement at each time point was implemented due the low solubility of the drug substance in the simulated vaginal fluid (˜16 ug/mL).

The results for both dose strengths followed the square root (t) model through 24 hours. The drug release was linear with time through at least 6 hours, and then began to taper off slightly by 24 hours. This demonstrated a typical release profile for a drug substance suspended in a viscoelastic matrix. The vessel-to-vessel variance (% RSD) was <4% for both runs, and the R2 values for the SQRT(t) was >0.99 through 24 hours.

From the data collected through 24 hours the drug release versus time for the applied dose (˜0.4 g) was estimated according to the Higuchi square root equation based on the total surface area of the IVRT membrane, and the average surface area of the vagina (see, P. Pendergrass, M Belovicz. Surface Area of the Human Vagina as Measured from Vinyl Polysiloxane Casts. February 2003. Gynecologic and Obstetric Investigation 55(2):110-3, incorporated herein by reference in its entirety). The percent drug release at T=24 hours (4.3% and 2.4% respectively) was included in the estimate for comparison to the measured result to the regression model. Tables 6 and 7 below show the estimated relates for the 1% diclofenac gel and 3% diclofenac gel, respectively

TABLE 6
Estimated Release for the 1% Diclofenac Vaginal Gel

| | | | Est. In |
|---|---|---|---|
| | Total μg | IVRT hours | Vivo hours |
| % Release | Release | (1.77 cm\hat{ }2) | (87.5 cm\hat{ }2) |
| 100 | 4000 | 10272.3 | 4.2 |
| 80 | 3200 | 6574.2 | 2.7 |
| 50 | 2000 | 2568.1 | 1.1 |
| 25 | 1000 | 642.0 | 0.3 |
| 4.3 | 174 | 19.4 | 0.0 |

TABLE 7
Estimated Release for the 3% Diclofenac Vaginal Gel

| | | | Est. In |
|---|---|---|---|
| | Total μg | IVRT hours | Vivo hours |
| % Release | Release | (1.77 cm\hat{ }2) | (87.5 cm\hat{ }2) |
| 100 | 12000 | 46358.5 | 19.0 |
| 80 | 9600 | 29669.5 | 12.1 |
| 50 | 6000 | 11589.6 | 4.7 |
| 25 | 3000 | 2897.4 | 1.2 |
| 2.4 | 287 | 26.5 | 0.01 |

The estimated time assumes the parameters of the Higuchi model are maintained throughout the drug release and do not account for total dose depletion, pH changes, swelling of the matrix, or dissolution of the gel. As an estimate the relationship between the total dose and drug release time can be modeled as the change in dose squared:

$$RT 2 = {( \frac{Q 2}{Q 1} )}^{2} * RT 1$$

### Where:

RT is the release time
Q1 is the reference dose
Q2 is the new dose
Example: Change in 100% release time of a 1% gel for a 0.4 g dose to a 1 g dose based on a surface area of 87.5 cm².

$$RT = {( \frac{1. * 10000}{0.4 * 10000} )}^{2} * 4.2 hours = 26.3 hours$$

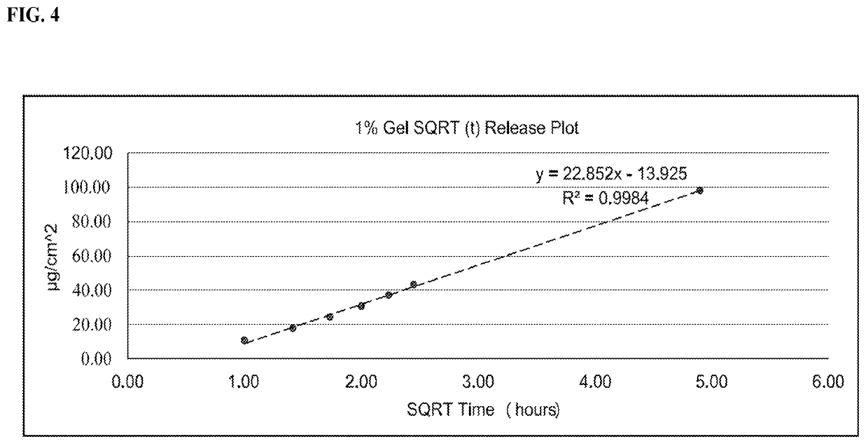

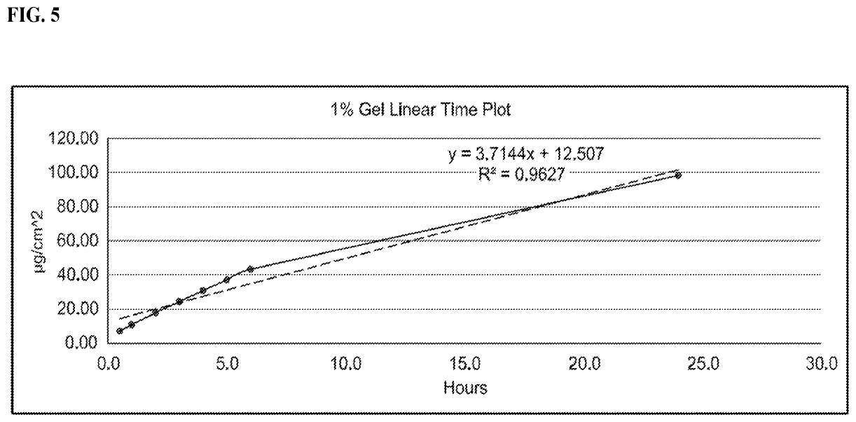

TABLE 8
IVRT Results for 1% w/w Diclofenac Gel (see also FIG. 4 and FIG. 5)

| | | | | | | | | | Avg. |
|---|---|---|---|---|---|---|---|---|---|
| | | | | | | | | | Slop |
| | sqrt | μg/cm² | | | | | | Avg. | (SQRT |
| Hours | (hrs) | 1 | 2 | 3 | 4 | 5 | 6 | μg/cm² | (t) |
| 0.5 | 0.71 | 7.13 | 6.96 | 8.85 | 6.53 | 5.53 | 7.34 | 7.06 | 22.3 |
| 1.0 | 1.00 | 10.61 | 10.51 | 13.11 | 10.59 | 8.45 | 11.21 | 10.75 | |
| 2.0 | 1.41 | 17.17 | 17.26 | 20.14 | 18.20 | 15.19 | 18.49 | 17.74 | |
| 3.0 | 1.73 | 23.39 | 23.51 | 26.76 | 25.33 | 21.61 | 25.23 | 24.30 | |
| 4.0 | 2.00 | 29.43 | 29.54 | 33.54 | 32.28 | 27.80 | 31.68 | 30.71 | |
| 5.0 | 2.24 | 35.50 | 35.50 | 40.22 | 39.21 | 33.79 | 38.01 | 37.04 | |
| 6.0 | 2.45 | 41.78 | 41.37 | 46.71 | 45.84 | 39.58 | 44.12 | 43.23 | |
| 24.0 | 4.90 | 96.08 | 93.23 | 100.52 | 103.37 | 95.95 | 100.18 | 98.22 | |

| | | | | | | | | |
|---|---|---|---|---|---|---|---|---|
| Linear Slope | 3.6 | 3.5 | 3.7 | 3.9 | 3.7 | 3.8 | 3.71 | |
| R² | 0.967 | 0.962 | 0.956 | 0.958 | 0.970 | 0.962 | | |
| SQRT (t) Slope | 21.8 | 21.1 | 22.4 | 23.7 | 22.2 | 22.7 | Linear Slope | 3.8 |
| | | | | | | | % RSD | |
| R² | 0.995 | 0.996 | 0.996 | 0.996 | 0.994 | 0.996 | SQRT(t) Slope | 3.9 |
| | | | | | | | % RSD | |

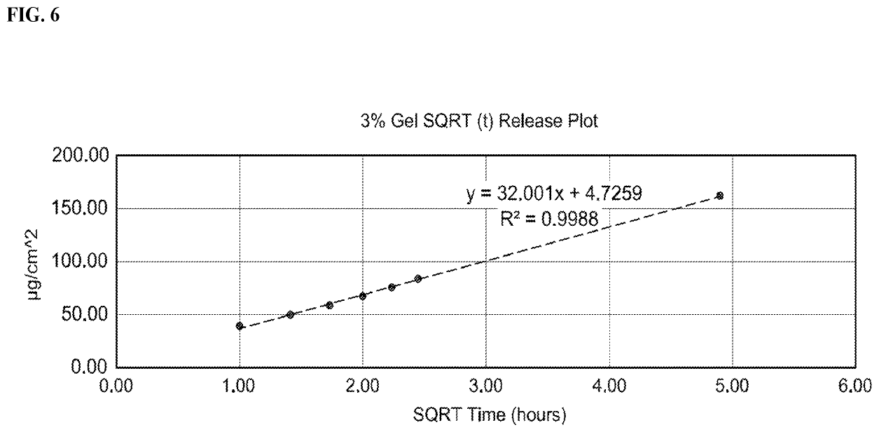

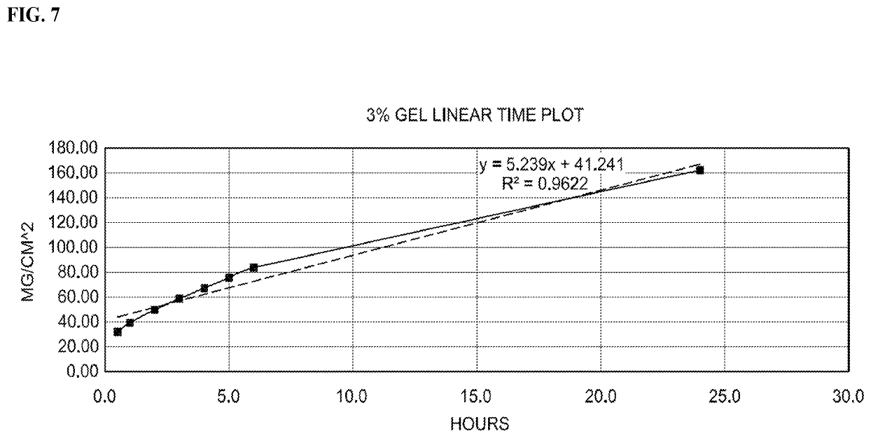

TABLE 9
IVRT Results for 3% w/w Diclofenac Gel (see also FIGs. 6 and 7)

| | | | | | | | | | Avg. |
|---|---|---|---|---|---|---|---|---|---|
| | sqrt | μg/cm\hat{ }2 | | | | | | | SQRT(t) |
| Hours | (hrs) | 1 | 2 | 3 | 4 | 5 | 6 | Avg. | Slope |
| 0.5 | 0.71 | 32.25 | 32.24 | 25.21 | 24.28 | 45.12 | 47.44 | 31.82 | 31.5 |
| 1.0 | 1.00 | 40.23 | 39.11 | 32.87 | 31.08 | 53.23 | 92.29 | 39.30 | |
| 2.0 | 1.41 | 51.22 | 48.89 | 43.60 | 41.66 | 63.28 | 129.12 | 49.73 | |
| 3.0 | 1.73 | 60.10 | 57.60 | 52.78 | 51.37 | 71.94 | 195.33 | 58.76 | |
| 4.0 | 2.00 | 68.68 | 65.73 | 61.55 | 59.95 | 80.32 | 265.81 | 67.25 | |
| 5.0 | 2.24 | 76.73 | 73.43 | 69.78 | 68.91 | 89.05 | 334.58 | 75.58 | |
| 6.0 | 2.45 | 84.76 | 81.15 | 77.64 | 77.22 | 97.69 | 407.06 | 83.69 | |
| 24.0 | 4.90 | 161.91 | 154.95 | 159.56 | 162.58 | 171.88 | 532.51 | 162.18 | |

| | | | | | | | | |
|---|---|---|---|---|---|---|---|---|
| Linear Slope | 5.2 | 4.9 | 5.4 | 5.6 | 5.1 | 18.4 | 5.2 | |
| R² | 0.958 | 0.962 | 0.965 | 0.969 | 0.956 | 0.709 | | |
| SQRT (t) Slope | 31.2 | 29.7 | 32.4 | 33.5 | 30.6 | 120.6 | Linear Slope | 5.1 |
| | | | | | | | % RSD | |
| R² | 0.999 | 0.998 | 0.997 | 0.996 | 0.998 | 0.873 | SQRT(t) Slope | 4.9 |
| | | | | | | | % RSD | |

### Example 6: Release Rate Evaluation

This study evaluated the release rate of diclofenac by in-vitro release testing of diclofenac 1% and 3% gel in simulated vaginal fluid. Two liters of simulated vaginal fluid was prepared and the release rate of diclofenac from 1% and 3% gels was evaluated. The formulation composition for simulated vaginal fluid is shown in Table 10 below. To prepare the simulated vaginal fluid, ˜1700 mL of purified water was transferred into a tared 2 L beaker with a stir bar, the components were mixed until dissolved, the pH was adjusted to 4.5 with 1.0N HCl/NaOH, the solution was transfer into a 2 L volumetric flask and diluted volumetrically with purified water and mix well, and the solution was degassed for 30 minutes prior to use.

TABLE 10
Formulation Composition of Simulated Vaginal Fluid

| | | | | Amount/ | Amount |
|---|---|---|---|---|---|
| Item | | | | Batch | Weighed |
| No. | Ingredient | % w/w | mg/mL | (g) | (g) |
| 1 | Sodium | 0.351 | 3.510 | 7.020 | |
| | Chloride | | | | |
| 2 | Potassium | 0.140 | 1.400 | 2.800 | |
| | Hydroxide | | | | |
| 3 | Calcium | 0.022 | 0.222 | 0.444 | |
| | Hydroxide | | | | |
| 4 | Bovine Serum | 0.002 | 0.018 | 0.036 | |
| | Albumin | | | | |
| 5 | Lactic Acid | 0.200 | 2.000 | 4.000 | |
| 6 | Acetic Acid | 0.100 | 1.000 | 2.000 | |
| 7 | Glycerol | 0.016 | 0.160 | 0.320 | |
| 8 | Urea | 0.040 | 0.400 | 0.800 | |
| 9 | D(+)-Glucose | 0.500 | 5.000 | 10.000 | |
| 10 | pH to 4.5 | QS | QS | QS | |
| | (HCl/NaOH) | | | | |
| 11 | Water | 98.629 | 986.29 | 1972.580 | |
| | Total | 100.000 | 1000.000 | 2000.000 | |

The results of the testing are shown in the tables below, where 178003-2201 corresponds to 1% w/w diclofenac gel, and 178003-2203 corresponds to 3% w/w/diclofenac gel.

TABLE 11
Release data for 178003-2201 (1% w/w diclofenac gel)

| | | | | | |
|---|---|---|---|---|---|
| | | Cell | mL of | Total μg | Total |
| | Measured | Volume | Sample | Previously | μg/cm\hat{ }2 |
| Cell | μg/mL | (mL) | collected | Removed | Released |

30 Minutes

| | | | | | |
|---|---|---|---|---|---|
| 1 | 0.63 | 20.0 | 19.521 | 0.00 | 7.13 |
| 2 | 0.62 | 20.0 | 19.353 | 0.00 | 6.96 |
| 3 | 0.78 | 20.0 | 19.666 | 0.00 | 8.85 |
| 4 | 0.58 | 20.0 | 19.427 | 0.00 | 6.53 |
| 5 | 0.49 | 20.0 | 17.948 | 0.00 | 5.53 |
| 6 | 0.65 | 20.0 | 19.407 | 0.00 | 7.34 |

60 Minutes

| | | | | | |
|---|---|---|---|---|---|
| 1 | 0.32 | 20.0 | 19.633 | 12.32 | 10.61 |
| 2 | 0.33 | 20.0 | 19.424 | 11.92 | 10.51 |
| 3 | 0.39 | 20.0 | 19.675 | 15.40 | 13.11 |
| 4 | 0.38 | 20.0 | 19.557 | 11.23 | 10.59 |
| 5 | 0.31 | 20.0 | 19.162 | 8.78 | 8.45 |
| 6 | 0.36 | 20.0 | 19.488 | 12.61 | 11.21 |

120 Minutes

| | | | | | |
|---|---|---|---|---|---|
| 1 | 0.59 | 20.0 | 19.589 | 18.66 | 17.17 |
| 2 | 0.61 | 20.0 | 19.371 | 18.41 | 17.26 |
| 3 | 0.63 | 20.0 | 19.568 | 23.07 | 20.14 |
| 4 | 0.68 | 20.0 | 19.473 | 18.58 | 18.20 |
| 5 | 0.61 | 20.0 | 19.450 | 14.70 | 15.19 |
| 6 | 0.65 | 20.0 | 19.565 | 19.65 | 18.49 |

180 Minutes

| | | | | | |
|---|---|---|---|---|---|
| 1 | 0.56 | 20.0 | 19.476 | 30.16 | 23.39 |
| 2 | 0.57 | 20.0 | 19.412 | 30.17 | 23.51 |
| 3 | 0.60 | 20.0 | 19.533 | 35.38 | 26.76 |
| 4 | 0.65 | 20.0 | 19.466 | 31.86 | 25.33 |
| 5 | 0.59 | 20.0 | 19.685 | 26.54 | 21.61 |
| 6 | 0.61 | 20.0 | 19.501 | 32.45 | 25.23 |

240 Minutes

| | | | | | |
|---|---|---|---|---|---|
| 1 | 0.55 | 20.0 | 19.476 | 41.10 | 29.43 |
| 2 | 0.55 | 20.0 | 19.368 | 41.27 | 29.54 |
| 3 | 0.61 | 20.0 | 19.472 | 47.08 | 33.54 |
| 4 | 0.63 | 20.0 | 19.484 | 44.50 | 32.28 |
| 5 | 0.56 | 20.0 | 19.682 | 38.06 | 27.80 |
| 6 | 0.59 | 20.0 | 19.461 | 44.36 | 31.68 |

300 Minutes

| | | | | | |
|---|---|---|---|---|---|
| 1 | 0.55 | 20.0 | 19.435 | 51.80 | 35.50 |
| 2 | 0.55 | 20.0 | 19.404 | 51.94 | 35.50 |
| 3 | 0.61 | 20.0 | 19.517 | 59.04 | 40.22 |
| 4 | 0.63 | 20.0 | 19.474 | 56.81 | 39.21 |
| 5 | 0.54 | 20.0 | 19.707 | 49.02 | 33.79 |
| 6 | 0.58 | 20.0 | 19.488 | 55.76 | 38.01 |

360 Minutes

| | | | | | |
|---|---|---|---|---|---|
| 1 | 0.57 | 20.0 | 19.300 | 62.52 | 41.78 |
| 2 | 0.54 | 20.0 | 19.435 | 62.52 | 41.37 |
| 3 | 0.59 | 20.0 | 19.566 | 70.90 | 46.71 |
| 4 | 0.60 | 20.0 | 19.523 | 69.08 | 45.84 |
| 5 | 0.52 | 20.0 | 19.714 | 59.64 | 39.58 |
| 6 | 0.56 | 20.0 | 19.524 | 66.99 | 44.12 |

24 hours

| | | | | | |
|---|---|---|---|---|---|
| 1 | 4.83 | 20.0 | NA | 73.54 | 96.08 |
| 2 | 4.61 | 20.0 | NA | 72.92 | 93.23 |
| 3 | 4.78 | 20.0 | NA | 82.43 | 100.52 |
| 4 | 5.11 | 20.0 | NA | 80.85 | 103.37 |
| 5 | 5.00 | 20.0 | NA | 69.91 | 95.95 |
| 6 | 4.98 | 20.0 | NA | 77.83 | 100.18 |

TABLE 12
Release data for 178003-2203 (3% w/w diclofenac gel)

| | | | | | |
|---|---|---|---|---|---|
| | | Cell | mL of | Total μg | Total |
| | Measured | Volume | Sample | Previously | μg/cm\hat{ }2 |
| Cell | μg/mL | (mL) | collected | Removed | Released |

30 Minutes

| | | | | | |
|---|---|---|---|---|---|
| 1 | 2.85 | 20.0 | 19.640 | 0.00 | 32.25 |
| 2 | 2.85 | 20.0 | 19.336 | 0.00 | 32.24 |
| 3 | 2.23 | 20.0 | 19.496 | 0.00 | 25.21 |
| 4 | 2.15 | 20.0 | 19.559 | 0.00 | 24.28 |
| 5 | 3.99 | 20.0 | 19.154 | 0.00 | 45.12 |
| 6 | 4.20 | 20.0 | 19.231 | 0.00 | 47.44 |

60 Minutes

| | | | | | |
|---|---|---|---|---|---|
| 1 | 0.76 | 20.0 | 19.628 | 56.05 | 40.23 |
| 2 | 0.70 | 20.0 | 19.380 | 55.17 | 39.11 |
| 3 | 0.73 | 20.0 | 19.621 | 43.50 | 32.87 |
| 4 | 0.65 | 20.0 | 19.399 | 42.03 | 31.08 |
| 5 | 0.89 | 20.0 | 19.644 | 76.48 | 53.23 |
| 6 | 4.13 | 20.0 | 19.410 | 80.73 | 92.29 |

120 Minutes

| | | | | | |
|---|---|---|---|---|---|
| 1 | 0.99 | 20.0 | 19.556 | 70.93 | 51.22 |
| 2 | 0.89 | 20.0 | 19.548 | 68.79 | 48.89 |
| 3 | 0.96 | 20.0 | 19.566 | 57.90 | 43.60 |
| 4 | 0.96 | 20.0 | 19.130 | 54.62 | 41.66 |
| 5 | 0.91 | 20.0 | 18.984 | 93.91 | 63.28 |
| 6 | 3.38 | 20.0 | 19.475 | 160.91 | 129.12 |

180 Minutes

| | | | | | |
|---|---|---|---|---|---|
| 1 | 0.81 | 20.0 | 19.594 | 90.21 | 60.10 |
| 2 | 0.79 | 20.0 | 19.401 | 86.13 | 57.60 |
| 3 | 0.83 | 20.0 | 19.558 | 76.76 | 52.78 |
| 4 | 0.90 | 20.0 | 19.317 | 72.91 | 51.37 |
| 5 | 0.81 | 20.0 | 19.562 | 111.09 | 71.94 |
| 6 | 5.95 | 20.0 | 19.476 | 226.76 | 195.33 |

240 Minutes

| | | | | | |
|---|---|---|---|---|---|
| 1 | 0.78 | 20.0 | 19.316 | 106.04 | 68.68 |
| 2 | 0.74 | 20.0 | 19.407 | 101.48 | 65.73 |
| 3 | 0.80 | 20.0 | 19.126 | 93.05 | 61.55 |
| 4 | 0.79 | 20.0 | 19.530 | 90.32 | 59.95 |
| 5 | 0.76 | 20.0 | 19.614 | 126.97 | 80.32 |
| 6 | 6.39 | 20.0 | 19.770 | 342.62 | 265.81 |

300 Minutes

| | | | | | |
|---|---|---|---|---|---|
| 1 | 0.74 | 20.0 | 19.519 | 121.03 | 76.73 |
| 2 | 0.70 | 20.0 | 19.464 | 115.89 | 73.43 |
| 3 | 0.76 | 20.0 | 19.136 | 108.26 | 69.78 |
| 4 | 0.81 | 20.0 | 19.544 | 105.74 | 68.91 |
| 5 | 0.79 | 20.0 | 19.817 | 141.88 | 89.05 |
| 6 | 6.16 | 20.0 | 19.651 | 469.01 | 334.58 |

360 Minutes

| | | | | | |
|---|---|---|---|---|---|
| 1 | 0.73 | 20.0 | 19.542 | 135.46 | 84.76 |
| 2 | 0.70 | 20.0 | 19.392 | 129.60 | 81.15 |
| 3 | 0.73 | 20.0 | 19.634 | 122.86 | 77.64 |
| 4 | 0.75 | 20.0 | 19.395 | 121.59 | 77.22 |
| 5 | 0.77 | 20.0 | 19.646 | 157.47 | 97.69 |
| 6 | 6.52 | 20.0 | 19.358 | 590.06 | 407.06 |

24 hours

| | | | | | |
|---|---|---|---|---|---|
| 1 | 7.23 | 18.9 | NA | 149.69 | 161.91 |
| 2 | 6.94 | 18.9 | NA | 143.21 | 154.95 |
| 3 | 7.88 | 18.4 | NA | 137.15 | 159.56 |
| 4 | 8.31 | 18.2 | NA | 136.22 | 162.58 |
| 5 | 6.93 | 19.0 | NA | 172.64 | 171.88 |
| 6 | 12.07 | 18.8 | NA | 716.31 | 532.51 |

### Example 7: Compatibility Assay of Diclofenac Gel in Gel Formulation 1

The results of analysis for the compatibility of 1%, 2%, and 3% Diclofenac Sodium Gel in Gel Formulation 1 composition are provided herein. The formulation composition of each Diclofenac Sodium Gel is outlined in Tables 13-15 below.

TABLE 13
Composition of 1% Diclofenac Sodium Gel Batch 178003-2201

| | | | | Amount/ |
|---|---|---|---|---|
| | | | | Batch |
| Item No. | Ingredient | % w/w | mg/g | (g) |
| 1 | Poloxamer F127 | 16.50 | 165.00 | 82.50 |
| 2 | Xanthan Gum, USP | 2.00 | 20.00 | 10.00 |
| 3 | Diclofenac Sodium | 1.00 | 10.00 | 5.00 |
| 4 | Citric Acid | 0.60 | 6.00 | 3.00 |
| | Monohydrate | | | |
| 5 | Sodium Citrate | 0.55 | 5.50 | 2.75 |
| | Dihydrate | | | |
| 6 | Benzyl Alcohol | 1.00 | 10.00 | 5.00 |
| 7 | Purified Water | 74.35 | 743.50 | 371.75 |
| 8 | Purified Water QS | 4.00 | 40.00 | 20.00 |
| 9 | Total | 100.00 | 1000.00 | 500.00 |

TABLE 14
Composition of 2% Diclofenac Sodium Gel Batch 178003-2202

| | | | | Amount/ |
|---|---|---|---|---|
| | | | | Batch |
| Item No. | Ingredient | % w/w | mg/g | (g) |
| 1 | Poloxamer F127 | 16.00 | 160.00 | 80.00 |
| | (Portion A) | | | |
| 2 | Poloxamer F127 | 0.50 | 5.00 | 2.50 |
| | (Portion B) | | | |
| 3 | Xanthan Gum, USP | 2.00 | 20.00 | 10.00 |
| 4 | Diclofenac Sodium | 2.00 | 20.00 | 10.00 |
| 5 | Citric Acid | 0.60 | 6.00 | 3.00 |
| | Monohydrate | | | |
| 6 | Sodium Citrate | 0.55 | 5.50 | 2.75 |
| | Dihydrate | | | |
| 7 | Benzyl Alcohol | 1.00 | 10.00 | 5.00 |
| 8 | Purified Water | 73.35 | 733.50 | 366.75 |
| 9 | Purified Water QS | 4.00 | 40.00 | 20.00 |
| 10 | Total | 100.00 | 1000.00 | 500.00 |

TABLE 15
Composition of 3% Diclofenac Sodium Gel Batch 178003-2203

| | | | | Amount/ |
|---|---|---|---|---|
| | | | | Batch |
| Item No. | Ingredient | % w/w | mg/g | (g) |
| 1 | Poloxamer F127 | 16.00 | 160.00 | 80.00 |
| | (Portion A) | | | |
| 2 | Poloxamer F127 | 0.5 | 5.00 | 2.50 |
| | (Portion B) | | | |
| 3 | Xanthan Gum, USP | 2.00 | 20.00 | 10.00 |
| 4 | Diclofenac Sodium | 3.00 | 30.00 | 15.00 |
| 5 | Citric Acid | 0.60 | 6.00 | 3.00 |
| | Monohydrate | | | |
| 6 | Sodium Citrate | 0.55 | 5.50 | 2.75 |
| | Dihydrate | | | |
| 7 | Benzyl Alcohol | 1.00 | 10.00 | 5.00 |
| 8 | Purified Water | 72.35 | 723.50 | 361.75 |
| 9 | Purified Water QS | 4.00 | 40.00 | 20.00 |
| 10 | Total | 100.00 | 1000.00 | 500.00 |

### Viscosity

The viscosity was evaluated using a calibrated parallel plate oscillating rheometer, model MCR 102 by Anton Paar, and a PP 25 measuring device. A single point viscosity was measured in duplicate at 0.22 1/s for 60 seconds at 25° C. and 37° C. Due to excessive air in the drug product a portion of each batch was centrifuged at 6,000 RPM for 10 minutes prior to measuring viscosity. The results outlined in 15 presents the average viscosity. Batches 178003-2201 and 178003-2202 exhibited a significant decrease in viscosity at 37° ° C. compared to the viscosity at 25° C. Batch 178003-2203 exhibited some thermo-reversible gel properties with a 17% increase in viscosity at 37° ° C.

The relationship (correlation) between the temperature and viscosity of the composition was important. Normally, viscosity of poloxamer gels increases with temperature. However, with the compositions described herein, the viscosity levels off or decreases. Without being bound by theory, it is believed this may be related to viscosity at room temperature (e.g., gel having higher viscosity at room temperature is more likely to have velocity decrease with increased temperature).

TABLE 16
Results for Viscosity at 0.22 1/s

| | | | Average | Average |
|---|---|---|---|---|
| | Shear | | Viscosity | Viscosity |
| | Rate | Duration | at 25° C. | at 37° C. |
| Batch No: | 1/s | Seconds | M*Pas | M*Pas |
| 178003-2201 | 0.22 | 60.0 | 1,652,400 | 996,155 |
| (1% diclofenac) | | | | |
| 178003-2202 | 0.22 | 60.0 | 895,255 | 827,575 |
| (2% diclofenac) | | | | |
| 178003-2203 | 0.22 | 60.0 | 953,035 | 1,135,450 |
| (3% diclofenac) | | | | |

pH

Due to the viscous nature of the drug product, pH was evaluated by diluting a portion of the drug product with an equal amount by weight of Total Organic Carbon (TOC) water and mixed until uniform. The pH results for each prototype are presented in Table 17, below. These results demonstrated a significant increase in pH with the increase in diclofenac sodium concentration. In some embodiments, this correlation may be due to the chemical nature of the ingredients.

Diclofenac solubility was shown to depend on pH, with higher solubility correlated with higher pH of the gel.

Due to the natural acidity of the vagina, vaginal compositions typically aim for a pH of about 4.5. Vaginal compositions described herein (e.g., using diclofenac) may have pH values up to about 5.5. In some embodiments, gels described herein containing diclofenac may require additional buffer (e.g., increased sodium citrate concentration) as compared to the gel containing other active agents.

TABLE 17
Results for pH

| Batch | pH |
|---|---|
| 178003-2201 | 5.04 |
| (1% diclofenac) | |
| 178003-2202 | 5.84 |
| (2% diclofenac) | |
| 178003-2203 | 6.54 |
| (3% diclofenac) | |

### Appearance

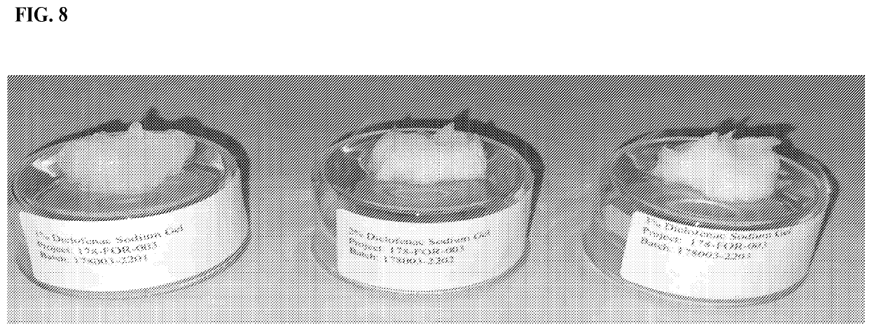

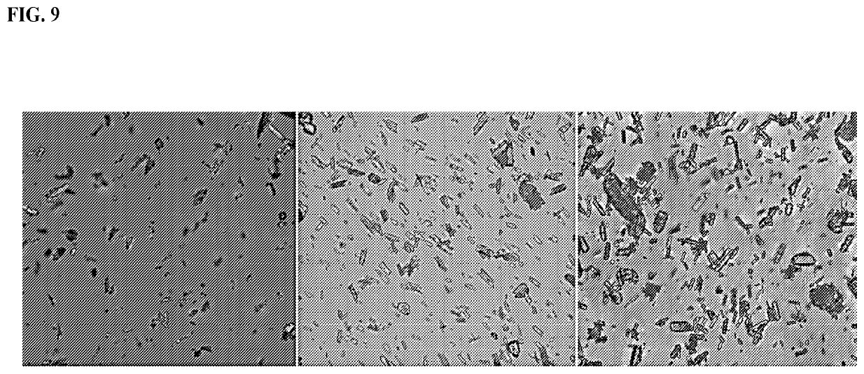

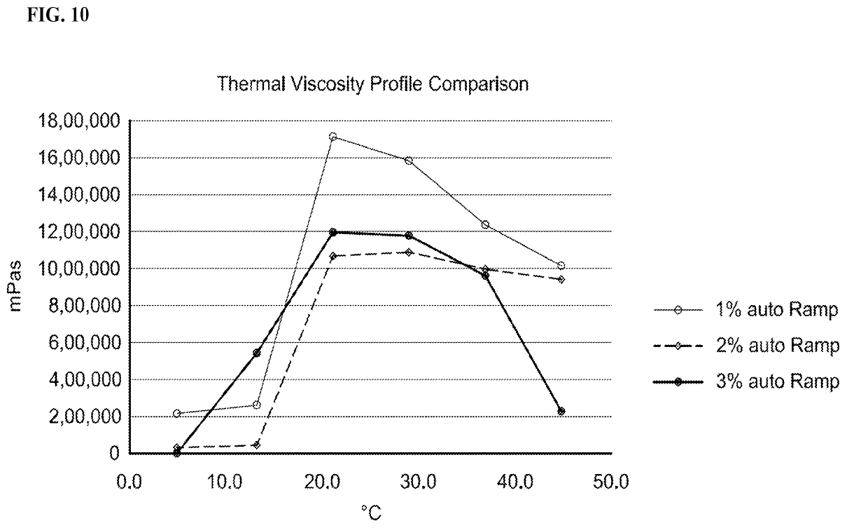

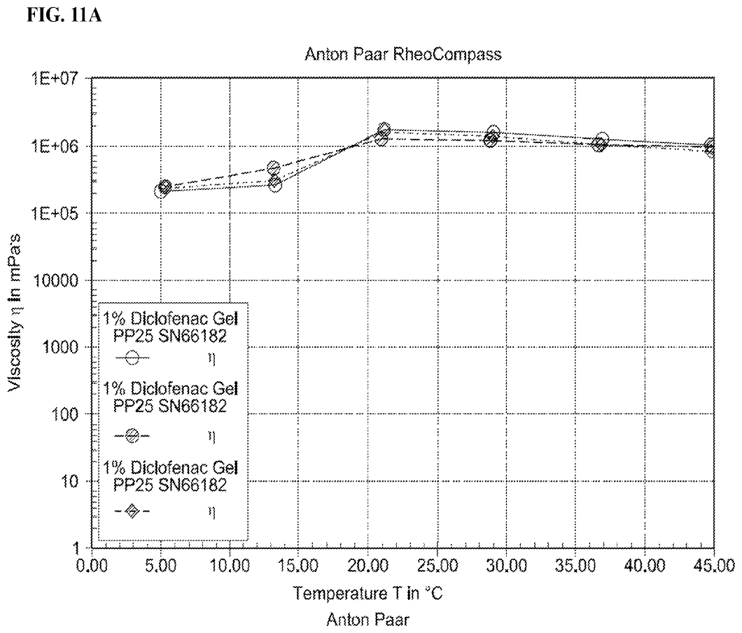

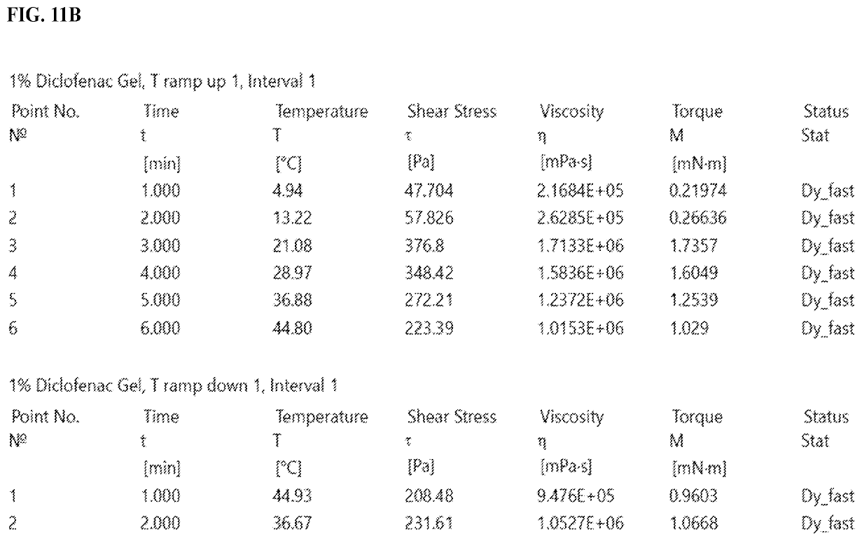

Appearance was evaluated visually based on color and general physical properties such as absence of agglomerates and absence of particulate matter. The results for appearance are presented in Table 18 and FIG. 8 and FIG. 9.

TABLE 18
Appearance results

| | Appearance | | | |
|---|---|---|---|---|
| | | | Absence of | |
| | | Absence of | Particulate | |
| Batch No. | Color | Agglomerates | Matter | Comments |
| 178003-2201 | Off White/Opaque | Conforms | Conforms | Contains excessive |
| (1% diclofenac) | | | | amounts of air |
| 178003-2202 | White/Slightly | Conforms | Conforms | Contains excessive |
| (2% diclofenac) | Opaque | | | amounts of air |
| 178003-2203 | White | Conforms | Conforms | Contains excessive |
| (3% diclofenac) | | | | amounts of air |

The rheological comparison between the three prototypes indicated that batch 178003-2203 exhibited some thermos-reversible gel properties with a 17% increase in the average viscosity at 37° C. Variation in pH was reported between prototypes but was expected due to the chemical nature of the API. Appearance results indicated a slight difference in color between prototypes with batch 178003-2203 exhibiting a more pronounced white due to the higher concentrations of API.

Based on these Data Diclofenac Sodium is Compatible with the Gel Formulation 1 Composition.

### Example 8: Uniformity and Viscosity

The results for assay of diclofenac sodium and benzyl alcohol were within the expected range. Uniformity for batches 2201 (1% diclofenac gel) and 2203 (3% diclofenac gel) was similar to the values obtained for assay and were within 2% RSD which is the verified precision of the method. Changes were observed in the rheology which correlated to the increased API (diclofenac) concentration. Particularly, these effects were observed in the shear stress of the flow point and in the thermal viscosity. These changes may be due to differences in the solids content and/or the water in the formulation. Surprisingly, a composition having diclofenac in suspension (not solution), was expected to take longer to release an equivalent amount of gel matrix. Also, a pH effect on release rate described herein. It is implicit in the data in Table 4 showing a non-linear variance in release rate as the API concentration goes up. When combined with the data in Table 17 that the pH goes up as the API concentration goes up, it supports the conclusion that higher pH supports faster release. Table 5 further supports this by showing that the 3% gel releases 50%, 80% and 100% faster than 1% or 2% diclofenac compositions.

### Uniformity

Uniformity was evaluated and the results are provided in Table 19 below

TABLE 19
uniformity results

| | Assay of | | Assay of | | |
|---|---|---|---|---|---|
| Batch | Diclofenac | | Benzyl | % Related | |
| Number | Sodium | Uniformity | Alcohol | Compounds | pH |
| 178003- | 99.4% | 96.0% | 105.5% | Indv. Imp: ND | 5.0 |
| 2201 | | 95.6% | | Total Imp: ND | |
| (1% | | 95.6% | | | |
| diclofenac) | % RSD | 0.2% | | | |
| 178003- | 97.0% | 90.4% | 102.6% | Indv. Imp: ND | 5.8 |
| 2202 | | 99.0% | | Total Imp: ND | |
| (2% | | 91.3% | | | |
| diclofenac) | % RSD | 5.1% | | | |
| 178003- | 96.7% | 96.3% | 102.4% | Indv. Imp: ND | 6.5 |
| 2203 | | 97.5% | | Total Imp: ND | |
| (3% | | 95.3% | | | |
| diclofenac) | % RSD | 1.1% | | | |

### Rheology

Testing was performed at 25° ° C., 10 rad/s, from 0.1-100% amplitude (logarithmic ramp) and results are shown in Table 20, below.

TABLE 20
Rheology

| | |
|---|---|
| LVER | Flow Point (Pa) |

| | LVER G′ | G′/G″ | Tau | G′ |
|---|---|---|---|---|
| Batch Number | (Pa) | (Pa) | (Pa) | (Pa) |
| 178003-2201 | 8790 | 9.1 | 307 | 2286 |
| (1% diclofenac) | | | | |
| 178003-2202 | 10100 | 9.1 | 276 | 2294 |
| (2% diclofenac) | | | | |
| 178003-2203 | 9930 | 11.0 | 215 | 2304 |
| (3% diclofenac) | | | | |

### Thermal Viscosity

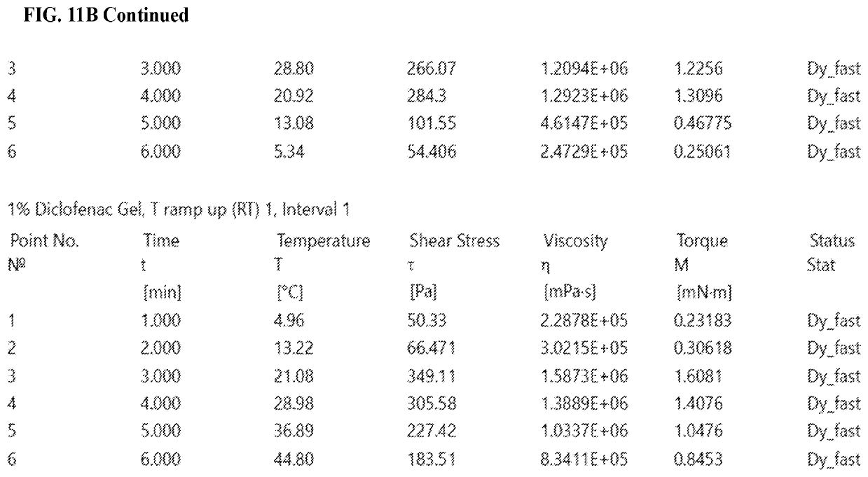

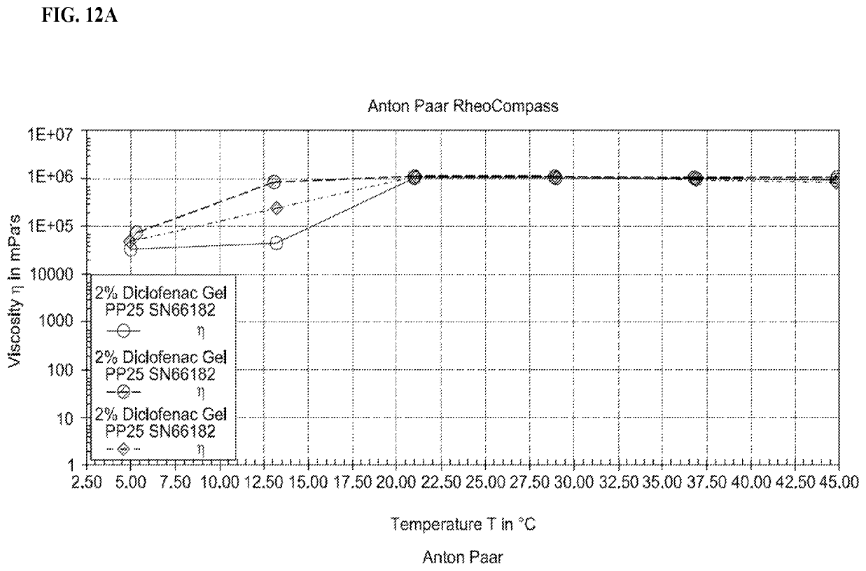

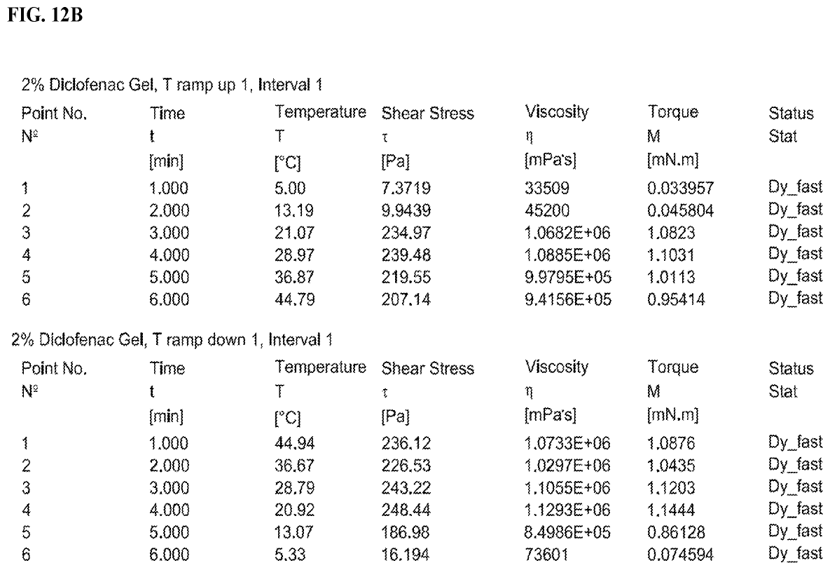

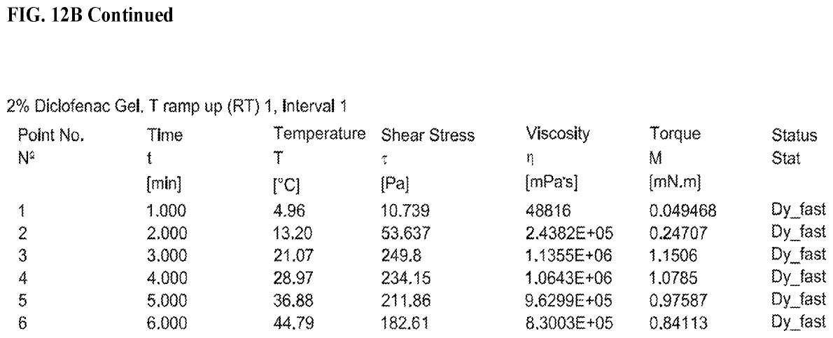

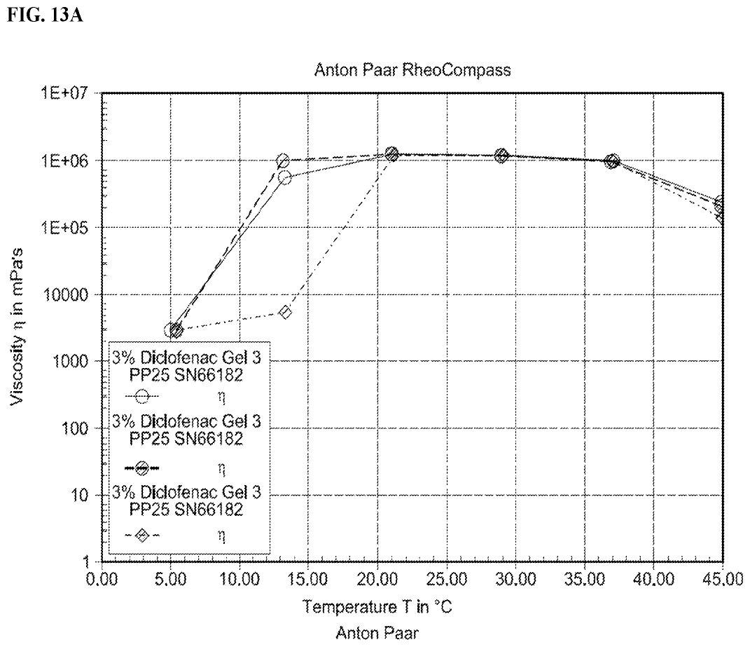

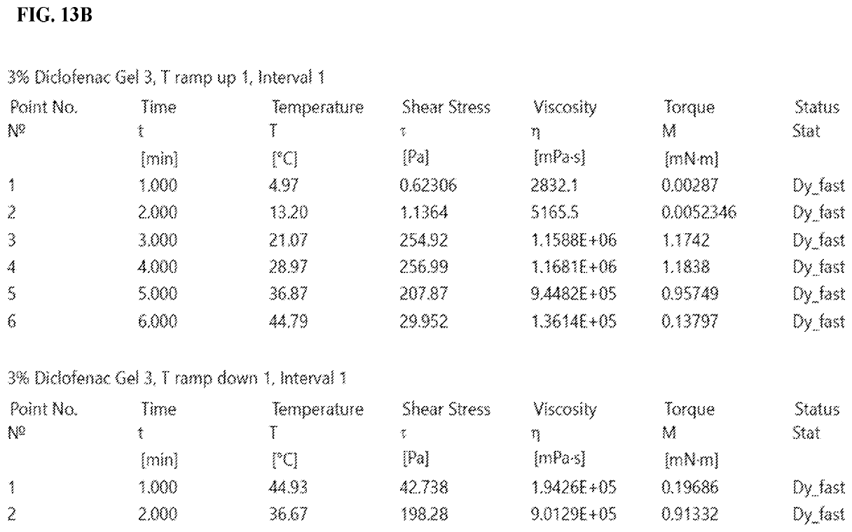

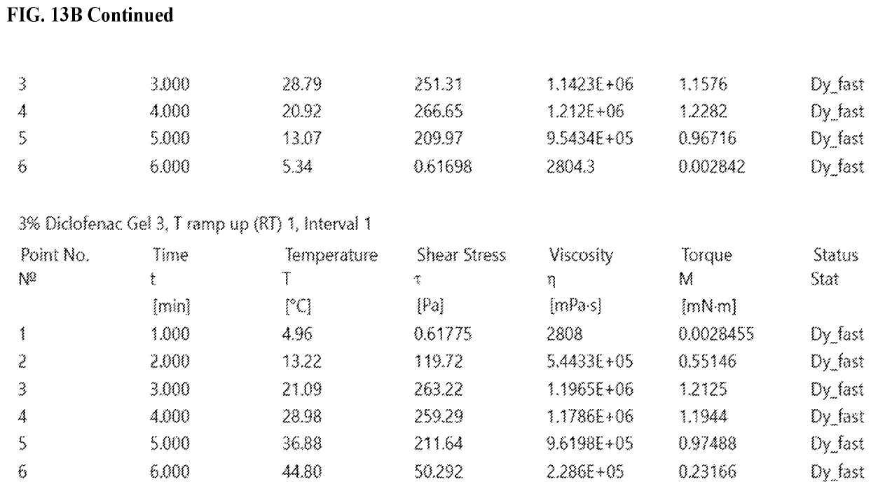

Thermal viscosity testing was performed at 0.221/s, 5° C.-45° ° C., at 1 minute intervals, pre shear 3 1/s at 60 seconds and results are shown in FIG X and Table 21 below. Thermal ramp up reports are shown in FIGS. 11A-11B, 12A-12B, and 13A-13B.

TABLE 21
Thermal viscosity

| | mPas | | |
|---|---|---|---|
| ° C. | 1% | 2% | 3% |
| 4.9 | 216,800 | 33,509 | 2,808 |
| 13.2 | 262,850 | 45,200 | 544,330 |
| 21.1 | 1,713,300 | 1,068,200 | 1,196,500 |
| 29.0 | 1,583,600 | 1,088,500 | 1,178,600 |
| 36.9 | 1,237,200 | 997,950 | 961,980 |
| 44.8 | 1,015,300 | 941,560 | 228,600 |

### Example 9: Air Removal

In this example, a manufacturing process is outlined describing air removal from the composition, as well as filing the composition into vaginal applicators is provided.

During mixing, 2000 g of benzyl alcohol and 33.0 kg of poloxamer 407 were added. Cooling and mixing continued until the solution was clear and the poloxamer was visually dissolved. The batch temperature reached approximately 11° C. during this step. This step created foam on the surface of the batch and the mixing speed was decreased to allow the foam to dissipate. Mixing speed was then increased, eventually reaching 1200 rpm for addition of the 4.00 kg of xanthan gum. After the addition of the xanthan gum, the product rapidly builds viscosity and the original mixing blade size proved inadequate. A switch was made to a 12-inch blade to better facilitate mixing at this stage of the process.

After this step in the process, the product generated a large amount of foaming. Initially, the product was transferred into a cooling, jacketed vacuum vessel and recirculated the product for several passes to help remove the air. A second approach used a Versator, which is a device especially designed to remove air from liquids and semi-solids. The product was passed through the Versator several times until all air was removed. Measurements of the viscosity after Versator treatment rose from 1.2 Cp (prior to use of the Versator) to over 3M Cp at room temperature.

Following this step, the product can be manually or filled using an automated filling machine. In some embodiments, the automated filling machine (e.g., a Capmatic automated filing machine) is especially designed to fill vaginal applicator-based products. As the viscosity of the product decreased at refrigerated temperatures, e.g., at temperatures of about 4° C. (e.g., a reduction in viscosity of 2-3×) provides a product suitable for automated filling into a vaginal applicator. In some embodiments, a cooled, jacketed vessel, feeding cooled product into the filler hopper to fill the applicators is used.

### Example 10: pH Effects on Release Rate

The purpose of this study is to evaluate effect of the matrix pH on the release rate (diffusion) of diclofenac from the drug product.

### Objectives

Prepare 3×100 g batches of Diclofenac 3% Gel at pH 3.5, 5.5, and 6.5
Evaluate the release rate of each pH adjusted batch by IVRT as per the test method RD-0240 over 24 hours.
Perform IVRT in simulated vaginal fluid for pH 3.5 and 5.5 over 24 hours
Determine the effects of pH on the release rate of Diclofenac Vaginal Gel.

### Study Design

| Spl. ID# | Receptor Media | Drug Product pH |
|---|---|---|
| 2215-01 | 50 mM Phosphate Buffer pH 7.0 | 3.5 |
| 2215-02 | 50 mM Phosphate Buffer pH 7.0 | 5.5 |
| 2215-03 | 50 mM Phosphate Buffer pH 7.0 | 6.5 |
| 2215-04 | Simulated Vaginal Fluid pH 4.5 | 3.5 |
| 2215-05 | Simulated Vaginal Fluid pH 4.5 | 6.5 |

Testing
Assay of Diclofenac: 90.0-110.0% of LC
IVRT: Report Release Rate μg/hr/cm²
Rheology: Thermal Rheology, Amplitude Sweep, Flow Curve
pH: Report Results
Appearance: Report Results

It was expected that the 2% diclofenac composition would release about 2× the amount of drug at a given time compared to the 1%, or conversely 3× that of the 3%. Surprisingly, however this was not observed. The higher concentration released much faster, and in fact the 2% diclofenac composition released all the active component faster than the 1% diclofenac composition released all of its active component. Without wishing to be bound by theory, it is contemplated that increases in pH increase the release rate of diclofenac.

### Definitions

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When the word “about” is used herein in reference to a number, it should be understood that still another embodiment of the disclosure includes that number not modified by the presence of the word “about.”

“Solution” according to the current disclosure is a clear, homogeneous liquid dosage form that contains one or more chemical substances dissolved in a solvent or mixture of mutually miscible solvents. A solution is a liquid preparation that contains one or more dissolved chemical substances in a suitable solvent or mixture of mutually miscible solvents. Because molecules of a drug substance in solution are uniformly dispersed, the use of solutions as dosage forms generally provides assurance of uniform dosage upon administration and good accuracy when the solution is diluted or otherwise mixed.

“Suspension” as used herein is a liquid dosage form that contains solid particles dispersed in a liquid vehicle.

When ranges are given by specifying the lower end of a range separately from the upper end of the range, it will be understood that the range can be defined by selectively combining any one of the lower end variables with any one of the upper end variables that is mathematically possible. Where ranges are recited, it will be understood that any subrange or value within the recited ranges, including endpoints, is contemplated.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A composition, comprising:
a poloxamer;
a stabilization polymer; and
an active ingredient for treating dysmenorrhea,
wherein the composition has a viscosity at room temperature of at least 1.5 million cP.

2. The composition of claim 1, where the composition is a gel.

3. The composition of any one of claim 1 or 2, wherein the composition has a viscosity at room temperature of at least 2 million cP.

4. The composition of any one of claims 1-3, wherein the composition has a viscosity at room temperature of at least 3 million cP.

5. The composition of any one of claims 1-4, wherein the composition contains no other component that changes the viscosity of said composition at room temperature by more than +/−100,000 centipoise.

6. The composition of any one of claims 1-5, wherein the poloxamer is present at 10-20 wt %.

7. The composition of any one of claims 1-6, wherein the poloxamer comprises poloxamer 407.

8. The composition of any one of claims 1-7, wherein at least 90 wt % of the poloxamer is poloxamer 407.

9. The composition of any one of claims 1-8, wherein the poloxamer comprise Pluronic® F-127.

10. The composition of any one of claims 1-9, wherein the poloxamer has a weight average molecular weight between 9 kDa and 16 kDa.

11. The composition of any one of claims 1-10, wherein the poloxamer has a structure:
HO—[CH₂—CH₂—O]ₐ—[CH₂—CH(CH₃)—O]_{b}—[CH₂—CH₂—O]ₐ—H.

12. The composition of claim 11, wherein a:b is from 2:1 to 4:1.

13. The composition of any one of claim 11 or 12, wherein a is between 99 and 103.

14. The composition of any one of claims 11-13, wherein b is between 54 and 58.

15. The composition of any one of claims 11-14, wherein a is about 101.

16. The composition of any one of claims 11-15, wherein b is about 56.

17. The composition of any one of claims 1-16, wherein the composition comprises air at no more than 15 vol %.

18. The composition of any one of claims 1-17, wherein the composition comprises air at no more than 5 vol %.

19. The composition of any one of claims 1-18, wherein the composition comprises air at no more than 1 vol %.

20. The composition of any one of claims 1-19, wherein the composition is substantially free of air.

21. The composition of any one of claims 1-20, wherein the composition has a density of at least 1 g/cm³.

22. The composition of any one of claims 1-21, wherein the active ingredient comprises diclofenac and/or a pharmaceutically acceptable salt thereof.

23. The composition of claim 22, wherein the active ingredient comprises diclofenac potassium.

24. The composition of any one of claims 1-23, wherein the active ingredient is present at 1-5 wt %.

25. The composition of any one of claims 1-24, wherein the stabilization polymer comprises xanthan gum.

26. The composition of any one of claims 1-25, wherein the stabilization polymer is present at 1-5 wt %.

27. The composition of any one of claims 1-26, wherein the composition further comprises citrate and/or a citrate salt.

28. The composition of claim 27, wherein the citrate and/or a citrate salt comprises citric acid.

29. The composition of any one of claim 27 or 28, wherein the citrate and/or a citrate salt comprises citric acid monohydrate.

30. The composition of any one of claims 27-29, wherein the citrate and/or a citrate salt comprises sodium citrate.

31. The composition of any one of claims 27-30, wherein the citrate and/or a citrate salt comprises sodium citrate dihydrate.

32. The composition of any one of claims 27-31, wherein the citrate and/or a citrate salt is present at 0.5-2 wt %.

33. The composition of any one of claims 1-32, wherein the composition further comprises benzyl alcohol.

34. The composition of claim 33, wherein the benzyl alcohol is present at 0.5-5 wt %.

35. The composition of any one of claims 1-34, wherein the viscosity is determined using a rheometer.

36. The composition of any one of claims 1-35, wherein the viscosity is determined using a viscometer.

37. A method, comprising:
applying, to a vagina of a subject, a composition comprising a poloxamer, a stabilization polymer, and an active ingredient for inducing cervical ripening, wherein the composition, as applied, has a viscosity of at least 1.5 million cP.

38. The method of claim 37, wherein the active ingredient comprises a prostaglandin and/or a pharmaceutically acceptable salt thereof.

39. The method of claim 38, wherein the prostaglandin comprises dinoprostone and/or a salt thereof.

40. The method of any one of claims 37-39, wherein the subject is human.

41. The method of any one of claims 37-40, where the composition is a gel.

42. The method of any one of claims 37-41, wherein the poloxamer comprises poloxamer 407.

43. The method of any one of claims 37-42, wherein the stabilization polymer comprises xanthan gum.

44. The method of any one of claims 37-43, wherein the composition comprises air at no more than 15 vol %.

45. The method of any one of claims 37-44, comprising applying the composition through an applicator to the vagina.

46. A method, comprising:
providing a composition comprising a poloxamer, a stabilization polymer, and an active ingredient for inducing cervical ripening; and
removing air from the composition.

47. The method of claim 46, wherein the active ingredient comprises a prostaglandin and/or a pharmaceutically acceptable salt thereof.

48. The method of claim 47, wherein the prostaglandin comprises dinoprostone and/or a salt thereof.

49. The method of any one of claims 46-48, wherein removing air comprises exposing the composition to a pressure of less than 100 mbar (absolute).

50. The method of any one of claims 46-49, wherein removing air comprises exposing the composition to a pressure of less than 50 mbar (absolute).

51. The method of any one of claims 46-50, wherein removing air comprises exposing the composition to a pressure of less than 40 mbar (absolute).

52. The method of any one of claims 49-51, comprising exposing the composition to the pressure for at least 30 min.

53. The method of any one of claims 46-52, wherein removing air comprises centrifuging the composition at 100 RPM.

54. The method of claim 53, comprising centrifuging the composition for at least 30 min.

55. The method of any one of claims 46-54, comprising removing air such that the composition comprises no more than 15 vol % air.

56. The method of any one of claims 46-55, wherein the composition has a viscosity at room temperature of at least 1.5 million cP.

57. The method of any one of claims 46-56, wherein the poloxamer comprises poloxamer 407.

58. The method of any one of claims 46-57, wherein the stabilization polymer comprises xanthan gum.

59. A method, comprising:
providing a composition comprising a poloxamer, a stabilization polymer, and an active ingredient for inducing cervical ripening; and
exposing the composition to a pressure of less than 100 mbar (absolute) for at least 30 minutes to form a gel.

60. The method of claim 59, wherein the active ingredient comprises a prostaglandin and/or a pharmaceutically acceptable salt thereof.

61. The method of claim 59, wherein the prostaglandin comprises dinoprostone and/or a salt thereof.

62. The method of any one of claims 59-61, wherein the gel has a viscosity at room temperature of at least 1.5 million cP.

63. The method of any one of claims 59-62, comprising exposing the composition to the pressure of less than 100 mbar until the gel comprises no more than 15 vol % air.

64. The method of any one of claims 59-63, wherein the poloxamer comprises poloxamer 407.

65. The method of any one of claims 59-64, wherein the stabilization polymer comprises xanthan gum.

66. A composition, comprising:
a poloxamer;
a stabilization polymer; and
an active ingredient for inducing cervical ripening,
wherein the composition is made by a process comprising forming a composition comprising the poloxamer, the stabilization polymer, and the active ingredient, and removing air from the composition.

67. The composition of claim 66, wherein the active ingredient comprises a prostaglandin and/or a pharmaceutically acceptable salt thereof.

68. The composition of claim 67, wherein the prostaglandin comprises dinoprostone and/or a salt thereof.

69. The composition of any one of claims 66-68, where the composition is a gel.

70. The composition of any one of claims 66-69, wherein the composition has a viscosity at room temperature of at least 1.5 million cP.

71. The composition of any one of claims 66-70, wherein the poloxamer comprises poloxamer 407.

72. The composition of any one of claims 66-71, wherein the stabilization polymer comprises xanthan gum.

73. The composition of any one of claims 66-72, wherein the composition comprises air at no more than 15 vol %.

74. A composition, comprising:
a poloxamer;
a stabilization polymer; and
an active ingredient for inducing cervical ripening,
wherein the composition is made by a process comprising forming a composition comprising the poloxamer, the stabilization polymer, and the active ingredient, and exposing the composition to a pressure of less than 100 mbar (absolute) for at least 30 minutes.

75. The composition of claim 74, wherein the active ingredient comprises a prostaglandin and/or a pharmaceutically acceptable salt thereof.

76. The composition of claim 75, wherein the prostaglandin comprises dinoprostone and/or a salt thereof.

77. The composition of any one of claims 74-76, where the composition is a gel.

78. The composition of any one of claims 74-77, wherein the composition has a viscosity at room temperature of at least 1.5 million cP.

79. The composition of any one of claims 74-78, wherein the poloxamer comprises poloxamer 407.

80. The composition of any one of claims 74-79, wherein the stabilization polymer comprises xanthan gum.

81. The composition of any one of claims 74-80, wherein the composition comprises air at no more than 15 vol %.

82. A composition for inducement of cervical ripening, wherein at least 90 wt % of the composition consists essentially of:
a poloxamer;
a stabilization polymer;
an active ingredient for inducing cervical ripening; and
water.

83. The composition of claim 82, wherein the active ingredient comprises a prostaglandin and/or a pharmaceutically acceptable salt thereof.

84. The composition of claim 82, wherein the prostaglandin comprises dinoprostone and/or a salt thereof.

85. The composition of any one of claims 82-84, wherein at least 75 wt % of the composition is water.

86. The composition of any one of claims 82-85, wherein at least 95 wt % of the composition consists essentially of poloxamer 407, xanthan gum, dinoprostone and/or a salt thereof, citrate and/or a citrate salt, and benzyl alcohol.

87. The composition of any one of claims 82-86, wherein at least 99 wt % of the composition consists essentially of poloxamer 407, xanthan gum, dinoprostone and/or a salt thereof, citric acid, sodium citrate, and benzyl alcohol.

88. The composition of any one of claims 82-87, where the composition is a gel.

89. The composition of any one of claims 82-88, wherein the composition has a viscosity at room temperature of at least 1.5 million cP.

90. The composition of any one of claims 82-89, wherein the poloxamer comprises poloxamer 407.

91. The composition of any one of claims 82-90, wherein the stabilization polymer comprises xanthan gum.

92. The composition of any one of claims 82-91, wherein the composition comprises air at no more than 15 vol %.

93. A composition, comprising:
a poloxamer;
a stabilization polymer; and
an active ingredient for inducing cervical ripening,
wherein the composition has a viscosity at room temperature of at least 1.5 million cP.

94. The composition of claim 93, where the composition is a gel.

95. The composition of any one of claim 93 or 94, wherein the composition has a viscosity at room temperature of at least 2 million cP.

96. The composition of any one of claims 93-95, wherein the composition has a viscosity at room temperature of at least 3 million cP.

97. The composition of any one of claims 93-96, wherein the composition contains no other component that changes the viscosity of said composition at room temperature by more than +/−100,000 centipoise.

98. The composition of any one of claims 93-97, wherein the poloxamer is present at 10-20 wt %.

99. The composition of any one of claims 93-98, wherein the poloxamer comprises poloxamer 407.

100. The composition of any one of claims 93-99, wherein at least 90 wt % of the poloxamer is poloxamer 407.

101. The composition of any one of claims 93-100, wherein the poloxamer comprise Pluronic® F-127.

102. The composition of any one of claims 93-101, wherein the poloxamer has a weight average molecular weight between 9 kDa and 16 kDa.

103. The composition of any one of claims 93-102, wherein the poloxamer has a structure:
HO—[CH₂—CH₂—O]ₐ—[CH₂—CH(CH₃)—O]_{b}—[CH₂—CH₂—O]ₐ—H.

104. The composition of claim 103, wherein a:b is from 2:1 to 4:1.

105. The composition of any one of claim 103 or 104, wherein a is between 99 and 103.

106. The composition of any one of claims 103-105, wherein b is between 54 and 58.

107. The composition of any one of claims 103-106, wherein a is about 101.

108. The composition of any one of claims 103-107, wherein b is about 56.

109. The composition of any one of claims 93-108, wherein the composition comprises air at no more than 15 vol %.

110. The composition of any one of claims 93-109, wherein the composition comprises air at no more than 5 vol %.

111. The composition of any one of claims 93-110, wherein the composition comprises air at no more than 1 vol %.

112. The composition of any one of claims 93-111, wherein the composition is substantially free of air.

113. The composition of any one of claims 93-112, wherein the composition has a density of at least 1 g/cm³.

114. The composition of any one of claims 93-113, wherein the active ingredient comprises a prostaglandin and/or a pharmaceutically acceptable salt thereof.

115. The composition of any one of claims 93-114, wherein the active ingredient is present at 1-5 wt %.

116. The composition of claim 114, wherein the prostaglandin comprises dinoprostone and/or a salt thereof.

117. The composition of claim 22, wherein the active ingredient comprises diclofenac sodium.

118. The composition of any one of claims 93-117, wherein the stabilization polymer comprises xanthan gum.

119. The composition of any one of claims 93-118, wherein the stabilization polymer is present at 1-5 wt %.

120. The composition of any one of claims 93-119, wherein the composition further comprises citrate and/or a citrate salt.

121. The composition of claim 120, wherein the citrate and/or a citrate salt comprises citric acid.

122. The composition of any one of claim 120 or 121, wherein the citrate and/or a citrate salt comprises citric acid monohydrate.

123. The composition of any one of claims 120-122, wherein the citrate and/or a citrate salt comprises sodium citrate.

124. The composition of any one of claims 120-123, wherein the citrate and/or a citrate salt comprises sodium citrate dihydrate.

125. The composition of any one of claims 120-124, wherein the citrate and/or a citrate salt is present at 0.5-2 wt %.

126. The composition of any one of claims 93-125, wherein the composition further comprises benzyl alcohol.

127. The composition of claim 126, wherein the benzyl alcohol is present at 0.5-5 wt %.

128. The composition of any one of claims 93-127, wherein the viscosity is determined using a rheometer.

129. The composition of any one of claims 93-128, wherein the viscosity is determined using a viscometer.

130. A method, comprising:
applying, to a vagina of a subject, a composition comprising a poloxamer, a stabilization polymer, and an active ingredient for treating dysmenorrhea, wherein the composition, as applied, has a viscosity of at least 1.5 million cP.

131. The method of claim 130, wherein the active ingredient comprises diclofenac.

132. The method of any one of claim 130 or 131, wherein the subject has dysmenorrhea.

133. The method of any one of claims 130-132, wherein the subject is at risk of dysmenorrhea.

134. The method of any one of claims 130-133, wherein the subject is human.

135. The method of any one of claims 130-134, where the composition is a gel.

136. The method of any one of claims 130-135, wherein the poloxamer comprises poloxamer 407.

137. The method of any one of claims 130-136, wherein the stabilization polymer comprises xanthan gum.

138. The method of any one of claims 130-137, wherein the composition comprises air at no more than 15 vol %.

139. The method of any one of claims 130-138, comprising applying the composition through an applicator to the vagina.

140. A method, comprising:
providing a composition comprising a poloxamer, a stabilization polymer, and an active ingredient for treating dysmenorrhea; and
removing air from the composition.

141. The method of claim 140, wherein the active ingredient comprises diclofenac.

142. The method of any one of claim 140 or 141, wherein removing air comprises exposing the composition to a pressure of less than 100 mbar (absolute).

143. The method of any one of claims 140-142, wherein removing air comprises exposing the composition to a pressure of less than 50 mbar (absolute).

144. The method of any one of claims 140-143, wherein removing air comprises exposing the composition to a pressure of less than 40 mbar (absolute).

145. The method of any one of claims 140-144, comprising exposing the composition to the pressure for at least 30 min.

146. The method of any one of claims 140-145, wherein removing air comprises centrifuging the composition at 100 RPM.

147. The method of claim 146, comprising centrifuging the composition for at least 30 min.

148. The method of any one of claims 140-147, comprising removing air such that the composition comprises no more than 15 vol % air.

149. The method of any one of claims 140-148, wherein the composition has a viscosity at room temperature of at least 1.5 million cP.

150. The method of any one of claims 140-149, wherein the poloxamer comprises poloxamer 407.

151. The method of any one of claims 140-150, wherein the stabilization polymer comprises xanthan gum.

152. A method, comprising:
providing a composition comprising a poloxamer, a stabilization polymer, and an active ingredient for treating dysmenorrhea; and
exposing the composition to a pressure of less than 100 mbar (absolute) for at least 30 minutes to form a gel.

153. The method of claim 152, wherein the active ingredient comprises diclofenac.

154. The method of any one of claim 152 or 153, wherein the gel has a viscosity at room temperature of at least 1.5 million cP.

155. The method of any one of claims 152-154, comprising exposing the composition to the pressure of less than 100 mbar until the gel comprises no more than 15 vol % air.

156. The method of any one of claims 152-155, wherein the poloxamer comprises poloxamer 407.

157. The method of any one of claims 152-156, wherein the stabilization polymer comprises xanthan gum.

158. A composition, comprising:
a poloxamer;
a stabilization polymer; and
an active ingredient for treating dysmenorrhea,
wherein the composition is made by a process comprising forming a composition comprising the poloxamer, the stabilization polymer, and the active ingredient, and removing air from the composition.

159. The composition of claim 158, wherein the active ingredient comprises diclofenac.

160. The composition of any one of claim 158 or 159, where the composition is a gel.

161. The composition of any one of claims 158-160, wherein the composition has a viscosity at room temperature of at least 1.5 million cP.

162. The composition of any one of claims 158-161, wherein the poloxamer comprises poloxamer 407.

163. The composition of any one of claims 158-162, wherein the stabilization polymer comprises xanthan gum.

164. The composition of any one of claims 158-163, wherein the composition comprises air at no more than 15 vol %.

165. A composition, comprising:
a poloxamer;
a stabilization polymer; and
an active ingredient for treating dysmenorrhea,
wherein the composition is made by a process comprising forming a composition comprising the poloxamer, the stabilization polymer, and the active ingredient, and exposing the composition to a pressure of less than 100 mbar (absolute) for at least 30 minutes.

166. The composition of claim 165, wherein the active ingredient comprises diclofenac.

167. The composition of any one of claim 165 or 166, where the composition is a gel.

168. The composition of any one of claims 165-167, wherein the composition has a viscosity at room temperature of at least 1.5 million cP.

169. The composition of any one of claims 165-168, wherein the poloxamer comprises poloxamer 407.

170. The composition of any one of claims 165-169, wherein the stabilization polymer comprises xanthan gum.

171. The composition of any one of claims 165-170, wherein the composition comprises air at no more than 15 vol %.

172. A composition for treatment of dysmenorrhea, wherein at least 90 wt % of the composition consists essentially of:
a poloxamer;
a stabilization polymer;
an active ingredient for treating dysmenorrhea; and
water.

173. The composition of claim 172, wherein the active ingredient comprises diclofenac.

174. The composition of any one of claim 172 or 173, wherein at least 75 wt % of the composition is water.

175. The composition of any one of claims 172-174, wherein at least 95 wt % of the composition consists essentially of poloxamer 407, xanthan gum, diclofenac and/or a salt thereof, citrate and/or a citrate salt, and benzyl alcohol.

176. The composition of any one of claims 172-175, wherein at least 99 wt % of the composition consists essentially of poloxamer 407, xanthan gum, diclofenac and/or a salt thereof, citric acid, sodium citrate, and benzyl alcohol.

177. The composition of any one of claims 172-176, where the composition is a gel.

178. The composition of any one of claims 172-177, wherein the composition has a viscosity at room temperature of at least 1.5 million cP.

179. The composition of any one of claims 172-178, wherein the poloxamer comprises poloxamer 407.

180. The composition of any one of claims 172-179, wherein the stabilization polymer comprises xanthan gum.

181. The composition of any one of claims 172-180, wherein the composition comprises air at no more than 15 vol %.

182. A method, comprising:
applying, to the vagina of a subject, a gel having a viscosity at room temperature of at least 1.5 million cP.

183. The method of claim 182, comprising applying the composition as a single dose.

184. The method of any one of claim 182 or 183, comprising applying the composition to the subject only once.

185. The method of any one of claim 182 or 183, comprising applying the composition to the subject no more than once every 30 days.

186. A composition comprising:
a poloxamer; and
a stabilization polymer,
wherein the composition has a viscosity at room temperature of at least 1.5 million cP.

187. A composition comprising:
a poloxamer;
a stabilization polymer, and
diclofenac,
wherein the composition has a viscosity at room temperature of at least 1.5 million cP.

188. A composition comprising:
a poloxamer;
a stabilization polymer, and
an active ingredient for inducing cervical ripening and/or treating dysmenorrhea and/or treating primary dysmenorrhea,
wherein the composition has a viscosity at room temperature of at least 1.5 million cP.

189. The composition of any one of claims 186-188, where the composition is a gel.

190. The composition of any one of claims 186-188, wherein the composition has a viscosity at room temperature of at least 2 million cP.

191. The composition of claim 190, wherein the composition has a viscosity at room temperature of at least 3 million cP.

192. The composition of any one of claims 186-191, wherein the composition contains no other component that changes the viscosity of said composition at room temperature by more than +/−100,000 centipoise.

193. The composition of any one of claims 186-192, wherein the poloxamer is present at 10-20 wt %.

194. The composition of any one of claims 186-193, wherein the poloxamer comprises poloxamer 407.

195. The composition of any one of claims 186-194, wherein at least 90 wt % of the poloxamer is poloxamer 407.

196. The composition of any one of claims 186-195, wherein the poloxamer comprises Pluronic® F-127.

197. The composition of any one of claims 186-196, wherein the poloxamer has a weight average molecular weight between 9 kDa and 16 kDa.

198. The composition of any one of claims 186-197, wherein the poloxamer has a structure:
HO—[CH₂—CH₂—O]ₐ—[CH₂—CH(CH₃)—O]_{b}—[CH₂—CH₂—O]ₐ—H.

199. The composition of claim 198, wherein a:b is from 2:1 to 4:1.

200. The composition of any one of claim 198 or 199, wherein a is between 99 and 103.

201. The composition of any one of claims 198-200, wherein b is between 54 and 58.

202. The composition of any one of claims 198-201, wherein a is about 101.

203. The composition of any one of claims 198-202, wherein b is about 56.

204. The composition of any one of claims 186-203, wherein the composition comprises air at no more than 15 vol %.

205. The composition of any one of claims 186-204, wherein the composition comprises air at no more than 5 vol %.

206. The composition of any one of claims 186-205, wherein the composition comprises air at no more than 1 vol %.

207. The composition of any one of claims 186-206, wherein the composition is substantially free of air.

208. The composition of any one of claims 186-207, wherein the composition has a density of at least 1 g/cm³.

209. A method for promoting cervical ripening in a subject comprising administering the composition of any one of claim 1-36, 66-129, 158-172, 174-181, or 186-208 to the vagina of the subject.

210. A method for treating dysmenorrhea in a subject comprising administering the composition of any one of claim 1-36, 66-129, 158-172, 174-181, or 186-208 to the vagina of the subject.

211. A kit comprising the composition of any one of claim 1-36, 66-129, 158-172, 174-181, or 186-208, wherein the kit includes an applicator suitable for vaginal application.

212. The kit of claim 211, wherein the applicator is pre-filled with the composition of any one of claim 1-36, 66-129, 158-172, 174-181, or 186-208.

213. The kit of claim 211, wherein the applicator is not pre-filled with the composition of any one of claim 1-36, 66-129, 158-172, 174-181, or 186-208.

214. The kit of 211, further comprising one or more of instructions for inserting the applicator into the vagina and instructions for inducing cervical ripening, for treating dysmenorrhea, treating menorrhagia, or promoting cervical ripening by applying the applicator filled with the composition.

215. The kit of claim 212, wherein the composition is at a temperature of about 4° C. for pre-filling the applicator.

216. A method, comprising:
applying, to a vagina of a subject, a composition comprising a poloxamer, a stabilization polymer, and an active ingredient for treating menorrhagia, wherein the composition, as applied, has a viscosity of at least 1.5 million cP.

217. The method of claim 216, wherein the active ingredient comprises diclofenac.

218. The method of any one of claim 216 or 217, wherein the subject has menorrhagia.

219. The method of any one of claims 216-218, wherein the subject is at risk of menorrhagia.

220. The method of any one of claims 216-219, wherein the subject is human.

221. The method of any one of claims 216-220, where the composition is a gel.

222. The method of any one of claims 216-221, wherein the poloxamer comprises poloxamer 407.

223. The method of any one of claims 216-222, wherein the stabilization polymer comprises xanthan gum.

224. The method of any one of claims 216-223, wherein the composition comprises air at no more than 15 vol %.

225. The method of any one of claims 216-224, comprising applying the composition through an applicator to the vagina.

226. A method, comprising:
providing a composition comprising a poloxamer, a stabilization polymer, and an active ingredient for treating menorrhagia; and
removing air from the composition.

227. The method of claim 226, wherein the active ingredient comprises diclofenac.

228. The method of any one of claim 226 or 227, wherein removing air comprises exposing the composition to a pressure of less than 100 mbar (absolute).

229. The method of any one of claims 226-228, wherein removing air comprises exposing the composition to a pressure of less than 50 mbar (absolute).

230. The method of any one of claims 226-229, wherein removing air comprises exposing the composition to a pressure of less than 40 mbar (absolute).

231. The method of any one of claims 226-230, comprising exposing the composition to the pressure for at least 30 min.

232. The method of any one of claims 226-231, wherein removing air comprises centrifuging the composition at 100 RPM.

233. The method of claim 232, comprising centrifuging the composition for at least 30 min.

234. The method of any one of claims 226-233, comprising removing air such that the composition comprises no more than 15 vol % air.

235. The method of any one of claims 226-234, wherein the composition has a viscosity at room temperature of at least 1.5 million cP.

236. The method of any one of claims 226-235, wherein the poloxamer comprises poloxamer 407.

237. The method of any one of claims 226-236, wherein the stabilization polymer comprises xanthan gum.

238. A method, comprising:
providing a composition comprising a poloxamer, a stabilization polymer, and an active ingredient for treating menorrhagia; and
exposing the composition to a pressure of less than 100 mbar (absolute) for at least 30 minutes to form a gel.

239. The method of claim 238, wherein the active ingredient comprises diclofenac.

240. The method of any one of claim 238 or 239, wherein the gel has a viscosity at room temperature of at least 1.5 million cP.

241. The method of any one of claims 238-240, comprising exposing the composition to the pressure of less than 100 mbar until the gel comprises no more than 15 vol % air.

242. The method of any one of claims 238-241, wherein the poloxamer comprises poloxamer 407.

243. The method of any one of claims 238-242, wherein the stabilization polymer comprises xanthan gum.

244. A method, comprising:
providing a composition comprising a poloxamer, a stabilization polymer, and an active ingredient for treating menorrhagia; and
exposing the composition to a pressure of less than 100 mbar (absolute) for at least 30 minutes to form a gel.

245. The method of claim 244, wherein the active ingredient comprises diclofenac.

246. The method of any one of claim 244 or 245, wherein the gel has a viscosity at room temperature of at least 1.5 million cP.

247. The method of any one of claims 244-246, comprising exposing the composition to the pressure of less than 100 mbar until the gel comprises no more than 15 vol % air.

248. The method of any one of claims 244-247, wherein the poloxamer comprises poloxamer 407.

249. The method of any one of claims 244-248, wherein the stabilization polymer comprises xanthan gum.

250. A method for treating menorrhagia in a subject comprising administering the composition of any one of claim 1-36, 66-129, 158-172, 174-181, or 186-208 to the vagina of the subject.

251. A method for treating pelvic pain in a subject comprising administering the composition of any one of claim 1-36, 66-129, 158-172, 174-181, or 186-208 to the vagina of the subject.

252. The method of claim 251, wherein the pelvic pain is generalized pelvic pain, acute pelvic pain or chronic pelvic pain.

253. The method of claim 251 or 252, wherein the pelvic pain results from endometriosis, adenomyosis, ovulatory pain, ovarian cyst pain and/or pelvic muscle associated pain.

254. The method or composition of any one of the above claims, wherein the composition releases about 50% of the active ingredient in about 10 hours to about 50 hours.

255. The method or composition of any one of the above claims, wherein the composition releases about 80% of the active ingredient in about 30 hours to about 60 hours.

256. The method or composition of any one of the above claims, wherein the composition releases about 100% of the active ingredient in about 30 hours to about 80 hours.
